(19) United States
(12) Patent Application Publication
(10) Pub. No.: US 2020/0249219 A1
(43) Pub. Date: Aug. 6, 2020

(54) TEST DEVICE AND METHOD TO DETERMINE LOW TEMPERATURE THERMAL CRACKING OF COMPOSITE MATERIALS

(71) Applicant: Ohio University, Athens, OH (US)

(72) Inventor: Sang Soo Kim, Athens, OH (US)

(21) Appl. No.: 16/783,460

(22) Filed: Feb. 6, 2020

Related U.S. Application Data
(60) Provisional application No. 62/801,820, filed on Feb. 6, 2019.

(51) Int. Cl. G01N 33/42; C10C 3/00; G01N 25/16

(52) U.S. Cl. CPC G01N 33/42; G01N 25/16; C10C 3/002

(57) ABSTRACT

An apparatus and method of predicting or determining temperature for thermal cracking of a composite material. Such method includes: (1) reducing the temperature of a composite material along a range of temperatures from a first temperature to a second temperature; (2) measuring dimensional changes in the composite material at a plurality of temperature points along the range of temperatures to generate a curve related to values for the coefficient of thermal expansion for the composite material; and (3) determining the transition temperature for the composite material, the transition temperature being at the intersection of two asymptotes of the curve, wherein the transition temperature correlates to the thermal cracking temperature of the composite material.

### CROSS-REFERENCE TO RELATED APPLICATIONS

The present application claims benefit of the filing date of U.S. Provisional Patent Application Ser. No. 62/801,820, filed Feb. 6, 2019, the disclosure of which is hereby incorporated by reference herein in its entirety.

### FIELD OF THE INVENTION

The present invention is directed to devices and methods for determining the low temperature cracking of composite materials.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Low temperature thermal cracking of asphalt pavements in cold regions in the U.S. costs billions of dollars of taxpayers' money annually. Minimizing low temperature cracking is one of the major goals for many state departments of transportation (DOTs). However, thorough evaluation of low temperature thermal cracking prior to construction of asphalt pavement is difficult and typically not performed by state DOTs and/or contractors due to the absence of accurate and easy to use test devices and/or methods.

The U.S. government spent $50 million in asphalt research on a single study called the Strategic Highway Research Program (SHRP) that lasted between 1988 and 1993 to develop test methods to evaluate low temperature thermal cracking, and later paid for several major research studies to develop a Simple Performance Test (SPT). Unfortunately, none of these studies produced a test or a method that can be used by technicians at DOTs and contractors. Further, only a few select research institutions own the equipment and use that equipment mainly for research.

Others have attempted to solve these problems, limitations, and/or deficiencies. However, all those attempts (both in practice and in research) have suffered various drawbacks. In practice, for example, for almost all asphalt pavements being placed in the U.S., the low temperature cracking potential is estimated from the flexibility of asphalt binder only. However, this method ignores the relevant mixture properties and the effects of the other mixture components such as aggregates, recycled asphalt pavement (RAP), recycled asphalt shingle (RAS), and many other additives commonly used in asphalt paving practice.

In research, there are two approaches to predict the low temperature thermal cracking of an asphalt mixture: The first of these is an analytical approach, where relevant mixture properties are determined in a laboratory and entered into established relationships for the low temperature cracking damage prediction. The first thorough approach was presented by Roque et al. [“Thermal Cracking Performance and Design of Mixtures Using Superpave™,” Journal of the Association of Asphalt Paving Technologists, (1995), Volume 64, pp. 718-735], and many similar approaches have been introduced. From mechanistic point of view, the prediction of low temperature cracking requires knowing or measuring three properties of materials: (1) stiffness, (2) strength, and (3) the coefficient of thermal expansion (CTE). However, CTE was never measured in these approaches, and the determination of strength or fracture properties in these methods is not thorough. The prediction of low temperature cracking in all of these approaches, then, relies heavily on low temperature stiffness (rheology). Thus, these approaches cannot be, and are not, thorough—and overall, too many assumptions are built into the lengthy calculations of these approaches.

The second research approach involves torture tests, where an asphalt mixture is subjected to a field-like condition and the cracking temperature measured. The Thermal Stress Restrained Specimen Test (TSRST) and a test using an asphalt concrete cracking device (ACCD) are typical examples of such torture tests. Since the test is performed under field-like conditions, all material properties relevant to low temperature cracking are accounted for together with climatic effects. However, TSRST is a very lengthy test that requires lengthy operator time and special equipment. In a typical asphalt lab, production of a beam shape asphalt sample is possible. Special equipment is required to compact an asphalt mat. The mat is then sawed into desired sizes; drying; gluing to metal platens; testing one sample at a time; and requiring large amount of liquid nitrogen to too cool. ACCD is a much simpler test when compared to TSRST. However, it still needs slicing an asphalt mat in two, coring the middle to create a void, and making a notch. Each ACCD ring is instrumented to measure temperature and strain. The void of the core is fitted with an ACCD ring. As temperature drops, asphalt sample contracts and the ACCD prevents the contraction, inducing tensile stress within the sample. Continued cooling will cause eventual failure at the notch. ACCD can typically test four specimens at the same time.

Due to lack of proper test equipment, and the difficulty and long time needed for testing, thermal properties of asphalt mixtures and the components thereof have not been studied widely. And, as noted above, many present approaches do not consider all properties of all materials in an asphalt mixture—such as CTE. Instead, a predictive equation has been commonly used for estimation of asphalt mixture CTE. However, the current mixture CTE prediction equation is based on a solid composite model and cannot represent the viscoelastic behavior of asphalt mixtures. Further, the effects of asphalt additives, modifiers, and recycled materials commonly used in the current asphalt paving practice cannot be estimated by the equation.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

As described above, the coefficient of thermal expansion (CTE) is an important property influencing the low temperature performance of asphalt pavement. However, the current predictive equation commonly used for asphalt mixture CTE does not provide accurate measurements. To overcome this and other issues (such as those described above in the Background), the present inventor has now developed a simple and reliable device for measuring asphalt mixture CTE, which can be used to study thermal contraction behavior. As will be described in greater detail below, this device (and related methods) can be used to study thermal contraction behavior of various asphalt mixtures, and have revealed for the first time that the CTE of an asphalt mixture is a sigmoid function of temperature. Aggregate CTE has a direct influence on asphalt mixture CTE and low temperature performance. Unlike asphalt binder glass transition, the transition behavior of asphalt mixture CTE appears to be related to the stiffness of asphalt binder and localized cracking of asphalt binder under restrained conditions. Thus, the present inventor has developed a new predictive model for asphalt mixtures.

Aspects of the present invention thus include, but are not limited to, (1) a CTE device for CTE measurement of an asphalt mixture and (2) methods for determining the effects of aggregate and binder thermal properties on the mixture CTE and low temperature performance (including a new predictive model for asphalt mixtures).

In that regard, a CTE device in accordance with principles of the present invention can be used to determine the coefficient of thermal expansion/contraction (CTE) of asphalt mixtures. For example, it has been determined by the present inventor that CTE transition temperature (Tₜᵣ) measured in the CTE device is strongly related to the thermal cracking temperature (T_{cr}) of the asphalt mixture. The CTE device test is repeatable and easy to perform. It thus may be used as an asphalt mixture design and quality control/quality assurance test by government agencies, contractors, and others.

The CTE device test may be performed over a wide range of temperatures relevant to the low temperature thermal cracking of asphalt pavements. For example, in one embodiment, the CTE device test may be performed over a range of temperatures from +20° C. to −60° C. During a CTE device test, specimen deformations are measured using linear variable differential transducers and temperature detectors. For example, in one embodiment of the CTE device, two linear variable differential transducers (LVDTs) are placed substantially perpendicular to each other. In addition, chamber temperature, CTE device frame temperature, sample surface temperature, and sample interior temperature can be measured using four resistance temperature detectors (RTDs). In one embodiment, measurements may be taken every 60 seconds.

The CTE device can be used to determine CTE of various asphalt mixtures. In one method of use, the CTE device measures the dimensional changes of a test specimen in two mutually perpendicular diametric directions as the temperature is lowered or raised at a predetermined rate (typically 10° C./hour or 20° C./hour).

The CTE values of all asphalt mixture tested with the CTE device vary with temperature and show a distinct transition at a very low temperature, usually between −40° C. to −20° C. Further, the measured CTE values were different for cooling and heating. Up until the work of the present inventor, researchers had believed that the transition is the result of phase change from a liquid state at warm temperatures to a glassy state at low temperatures (consequently called a glass transition temperature—T_{g}); however, researchers had no explanation for the different CTE values for cooling and heating cycles.

The work of the present inventor described herein, however, determined that the transitions were the result of internal cracking induced by differential thermal contraction of asphalt binder and aggregate—and the present inventor also determined that this can be used for prediction of the low temperature thermal cracking of asphalt mixtures. Furthermore, the work of the present inventor determined that the different CTEs for cooling and heating were the phenomena caused by the development of internal cracks. The mechanism of internal damage of asphalt mixture at low temperatures is believed to be due to the differential thermal contractions. Asphalt mixture is a composite consisting of asphalt binder and mineral aggregates, where asphalt and aggregates are bonded together. While the CTE values for aggregates range between 5 με/° C. to 15 με/° C., the CTE values for asphalt binders are much larger with typical values ranging between 15 με/° C. to 300 με/° C. As temperature is lowered from an ambient temperature to a low temperature, asphalt binder placed between aggregates contracts more rapidly than the bonded aggregates, developing a tensile stress within the asphalt binder and pulling asphalt mixture inward (contraction). As the temperature continues to drop, the tensile stress within the asphalt binder continues to increase. Ultimately, asphalt binder starts to fracture at locations where the tensile stress reaches the failure strength. As the fracturing of asphalt binder continues at more locations, the inward stress that made the mixture contract is gradually reduced and some portion of already developed contraction recovers (expand), but very slowly due to high stiffness of asphalt binder at low temperatures. This new model developed by the present inventor also explains the extremely low CTE values of asphalt mixtures measured by the CTE device at very low temperatures, near −60° C. When sufficient level of asphalt binder cracking occurs, the whole asphalt mixture may experience very small contraction, causing a near-zero CTE.

And so, another aspect of the present invention is directed to a method of predicting or determining temperature for thermal cracking of a composite material. Such method includes: (1) reducing the temperature of a composite material along a range of temperatures from a first temperature to a second temperature; (2) measuring dimensional changes in the composite material at a plurality of temperature points along the range of temperatures to generate a curve related to values for the coefficient of thermal expansion for the composite material; and (3) determining the transition temperature for the composite material, the transition temperature being at the intersection of two asymptotes of the curve, wherein the transition temperature correlates to the thermal cracking temperature of the composite material.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is a photograph depicting a CTE device, in accordance with principles of the present invention, the CTE device including a frame within a temperature chamber, and having a test sample positioned within the frame.

FIG. 2 is a graph depicting test results from a sample in a CTE device with two consecutive cooling and heating cycles.

FIG. 3 is a schematic depicting the internal thermal cracking mechanism in a composite of aggregate and asphalt binder.

FIG. 4 is a graph showing the results from an experiment performed concerning the internal cracking of an asphalt mixture.

FIG. 5 is a graph showing the results from a second experiment performed concerning the internal cracking of an asphalt mixture.

FIG. 6A is a photograph showing sample preparation for an asphalt concrete cracking device (ACCD), and FIG. 6B is a photograph showing four ACCD samples.

FIG. 7 is a graph showing cracking temperature (T_{cr}) as measured in an ACCD versus transition temperature (Tₜᵣ) as determined with use of a CTE device.

FIG. 8 is a graph showing ACCD T_{cr} versus CTE device Tₜᵣ from literature [Kim, S., M. Nazzal, A. Abbas, M. Akentuna, and M. Arefin, “Evaluation of Low Temperature Cracking Resistance of WMA,” Final Report FHWA/OH-2015111 (2015)].

FIG. 9A is a photograph and FIG. 9B is a schematic of the use of a prior asphalt thermal cracking analyzer to measure CTE and Tₜᵣ [by Marasteanu, M., Zofka, A., Turos, M., Li, X., Velasquez, R., Li, X., Buttlar, W., Paulino, G., Braham, A., Dave, E., Ojo, J., Bahia, H., Williams, C., Bausano, J., Kvasnak, A., Gallistel, & A., McGraw, J. (2007) “Investigation of low temperature cracking in asphalt pavements,” Report No. MN/RC 2007-43. Minnesota Department of Transportation, St. Paul, Minn.].

FIGS. 10A and 10B are photographs of equipment for a thermal stress restrained specimen test (TSRST) for thermal cracking temperature measurement.

FIG. 11A is a graph depicting TSRST cracking temperature versus cooling transition temperature, and FIG. 11 B is a graph depicting TSRST cracking temperature versus heating transition temperature.

FIG. 12 is a graph depicting CTE device test results showing transition near −30° C. for a tested sample.

FIG. 13 is a graph depicting thermal strain data resulting from tests in a CTE device.

FIG. 14 is a graph depicting results from tests in a CTE device where plot of the data demonstrates CTE as a sigmoid function of temperature.

FIG. 15 is a graph depicting CTE transition rate.

FIG. 16 is a graph depicting CTE as a sigmoid function of temperature.

FIGS. 17A and 17B are graphs showing the CTE effects on cracking temperature (T_{cr}) as measure with an ACCD.

FIG. 18 is a graph depicting a theoretical calculation of CTE contribution in cracking temperature.

FIGS. 19A-19D are graphs depicting the results of tests of 8 mixtures in a CTE device.

FIG. 20 is a graph depicting the CTE difference between mixtures due to the particular aggregate used.

FIG. 21 is a graph depicting ACCD cracking temperature versus CTE of 8 specific mixtures tested.

FIG. 22 is a graph depicting the predicted CTE (by the Mechanistic-Empirical Pavement Design Guide—“MEPDG”) for each mixture plotted against the measured CTE for each mixture.

FIGS. 23A and 23B are graphs showing plots of binder CTE and mixture CTE for two of the binders (AAA-1 and AAM-1) and two mixtures [the two binders each mixed with Grove City (GC) Limestone mix].

FIG. 24 is a graph depicting measurements of volume change over temperature change for the various components of asphalt mixture (asphalt mixture including GC aggregate and AAA-1 binder).

FIG. 25 is a graph plotting binder stiffness at 60 sand mixture CTE against temperature for the two mixtures including AAA-1 binder and AAA-1 binder alone.

FIG. 26 is a plot of binder stiffness [bending beam rheometer (BBR) stiffness] versus mixture CTE (in a temperature range of 30° C. to −55° C., for the GC AAA-1 mixture).

FIG. 27 is a graph showing a polt of mixture CTE against binder stiffness, showing mixture CTE as a sigmoid function of S(60 s).

FIG. 28 is a graph showing a mixture CTE prediction by α_{agg}, α_{b}(T) and S(60 s) against measured mixture CTE.

FIG. 29 is a graph showing a mixture CTE prediction by α_{agg}, α₁ and S(60 s) against measured mixture CTE.

FIG. 30 is a graph showing a second mixture CTE prediction by α_{agg}, α₁ and S(60 s) against measured mixture CTE.

FIG. 31A is a graph showing test results for CTE versus temperature, and

FIG. 31 B is a graph showing a model prediction of CTE versus temperature.

FIG. 32 is a graph showing plots of predicted mixture CTE against measured mixture CTE for eight mixtures, and how those relate to a line of equality between prediction and measurement.

FIG. 33 is a schematic depicting the internal thermal cracking mechanism in a composite of aggregate and asphalt binder.

FIGS. 34A and 34B are graphs showing cracking temperature as measured with ACCD against mixture transition temperature. In FIG. 34A, the CTE test was run from 20° C. to −55° C. And in FIG. 34B, the CTE test was 20° C. to −35° C. The Tₜᵣ values are little variable; and mixtures includes hot mix asphalt (HMA), warm mix asphalt (WMA), reclaimed asphalt pavement (RAP), reclaimed asphalt shingles (RAS), styrene-butadiene-styrene (SBS) modified asphalt, polyphosphoric acid (PPA) modified asphalt, unmodified, LS, Gravel, Compaction Effort.

FIG. 35 is a graph showing thermal strain versus temperature plot for an asphalt mixture sample cooled to −60° C. and warmed to −20° C. repeatedly for four cycles.

FIG. 36 is a graph showing ice pressure against pipe surface temperature for various concentrations of CaCl₂.

FIGS. 37A and 37B are graphs showing the effect of salt (FIG. 37A) and saturation (FIG. 37B) on hot mix asphalt (HMA) damage (with freeze expansion measured by the CTE device).

FIG. 38A is a graph showing freeze expansion versus ice pressure, and

FIG. 38B shows indirect tensile strength against freezing expansion after one freezing cycle.

FIG. 39 is a graph showing the difference from chamber temperature of CTE device frame temperature (at two locations), sample surface temperature, and sample interior temperature (middle chamber) as measured using four resistance temperature detectors (RTDs) every 60 seconds as the chamber temperature is cooled from 20° C. to −60° C.

FIG. 40 is a second graph regarding temperatures inside the chamber of the CTE device, and plots sample strain over time against temperature.

FIG. 41A is a graph depicting measurements of volume change over temperature change for the various components of asphalt mixture (asphalt mixture including GC aggregate and AAA-1 binder); and FIG. 41B is a graph depicting measurements of volume change over temperature change for the various components of a second asphalt mixture (asphalt mixture including BC aggregate and AAA-1 binder).

FIG. 42A is a graph plotting binder stiffness at 60 sand mixture CTE against temperature for the two mixtures including AAA-1 binder and AAA-1 binder alone. FIG. 42B is a graph plotting binder stiffness at 60 sand mixture CTE against temperature for the two mixtures including AAC-1 binder and AAC-1 binder alone. FIG. 42C is a graph plotting binder stiffness at 60 sand mixture CTE against temperature for the two mixtures including AAF-1 binder and AAF-1 binder alone. FIG. 42D is a graph plotting binder stiffness at 60 sand mixture CTE against temperature for the two mixtures including AAM-1 binder and AAM-1 binder alone.

FIG. 43A is a graph showing chamber temperature of the CTE device over a 110 hour CTE experiment, and FIG. 43B is a graph showing microstrain against temperature for a PG 70-22M control mixture.

FIGS. 44A and 44B are graphs showing thermal strain above Tₜᵣ (FIG. 44A) and below Tₜᵣ (FIG. 44B). As can be seen in FIG. 44A, above Tₜᵣ, cooling CTE equals heating CTE, and this is repeatable. And, as can be seen in FIG. 44B, below Tₜᵣ, cooling CTE does not equal heating CTE, and heating strain is greater than cooling strain. This is also repeatable.

FIG. 45 is a graph plotting CTE against temperature and showing cooling and heating CTE by slope and B-A Eq.

FIG. 46 is a graph of thermal strain against temperature for cycles 4-7 of cooling and heating (to show implications of thermal and load fatigue, moisture). In restrained conditions, cracks occur at warmer temperatures and heal at warmer temperatures. Cracks get wider for each temperature cycle, and may be the cause of more cracks and potholes in early Spring.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described above, the coefficient of thermal expansion (CTE) is an important property influencing the low temperature performance of asphalt pavement. However, the current predictive equation commonly used for asphalt mixture CTE does not provide accurate measurements. To overcome this and other issues (such as those described above in the Background), the present inventor has now developed a simple and reliable device for measuring asphalt mixture CTE, which can be used to study thermal contraction behavior. As will be described in greater detail below, this device (and related methods) can be used to study thermal contraction behavior of various asphalt mixtures, and have revealed for the first time that the CTE of an asphalt mixture is a sigmoid function of temperature. Aggregate CTE had a direct influence on asphalt mixture CTE and low temperature performance. Unlike asphalt binder glass transition, the transition behavior of asphalt mixture CTE appears to be related to the stiffness of asphalt binder and localized cracking of asphalt binder under restrained conditions. Thus, the present inventor has developed a new predictive model for asphalts (e.g., dense graded hot mix asphalts).

Aspects of the present invention thus include, but are not limited to, (1) a CTE device for CTE measurement of an asphalt mixture and (2) methods for determining the effects of aggregate and binder thermal properties on the mixture CTE and low temperature performance (including a new predictive model for asphalt mixtures).

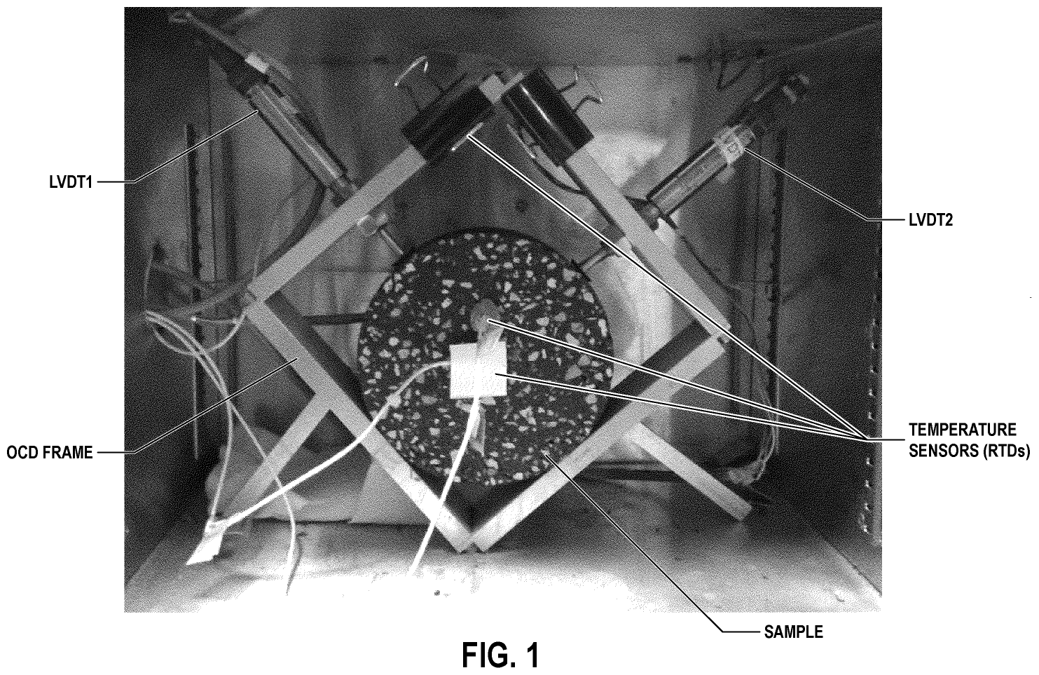

In that regard, an illustrated example of a CTE device in accordance with principles of the present invention is shown in FIG. 1. Such a device can be used to determine the coefficient of thermal expansion/contraction (CTE) of asphalt mixtures. For example, it has been determined by the present inventor that CTE transition temperature (Tₜᵣ) measured in the CTE device is strongly related to the thermal cracking temperature (T_{cr}) of the asphalt mixture. The CTE device test is repeatable and easy to perform. It thus may be used as an asphalt mixture design and quality control/quality assurance test by government agencies, contractors, and others.

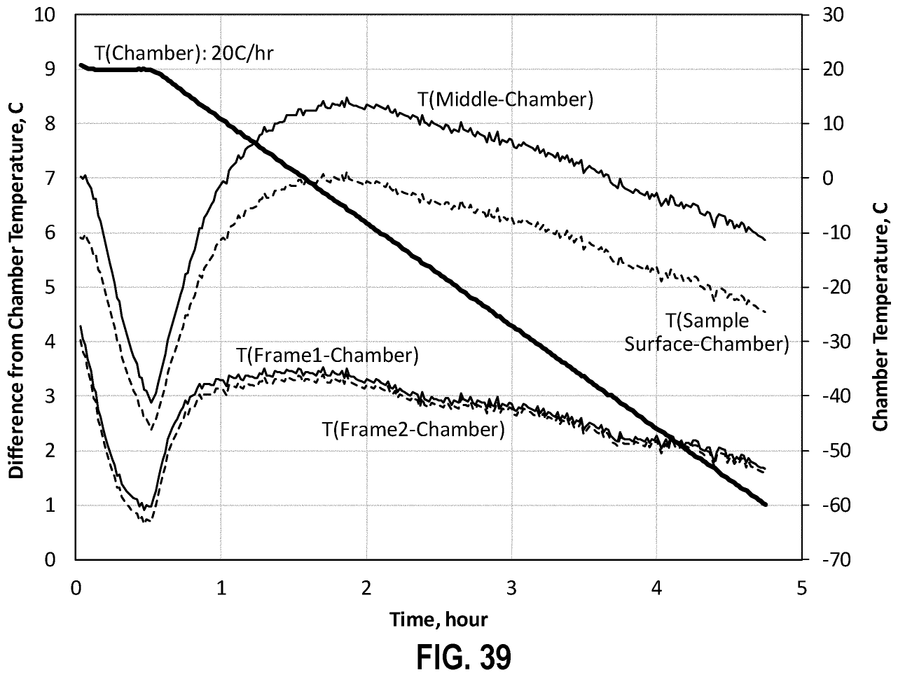

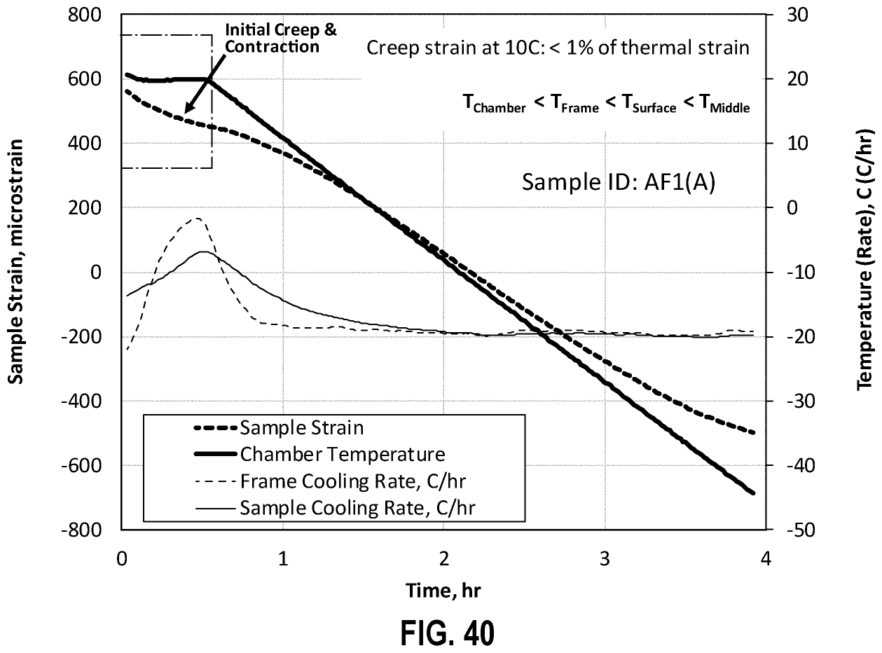

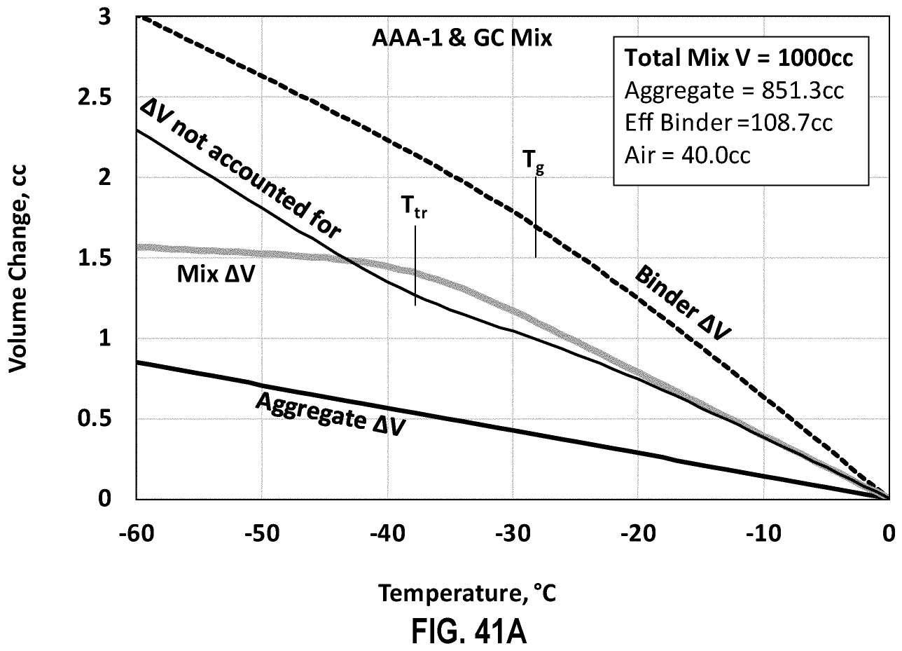

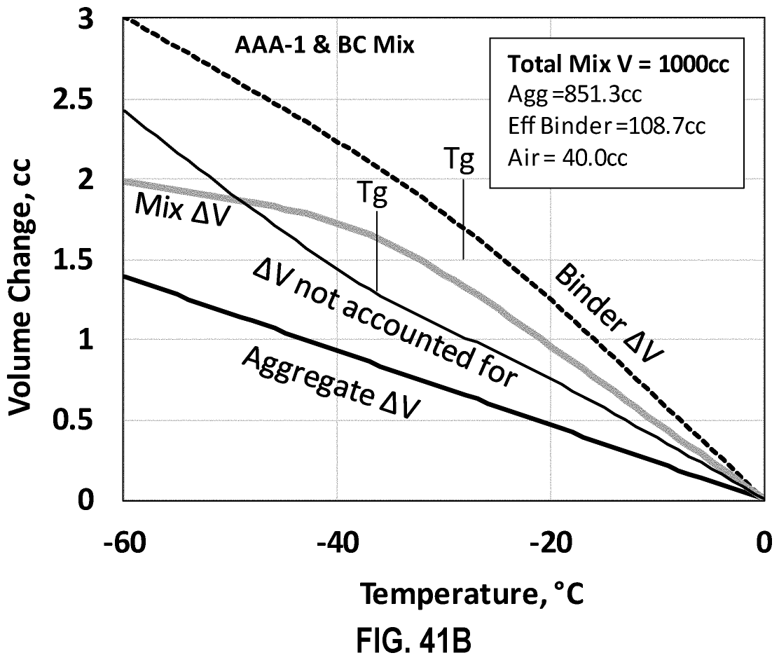

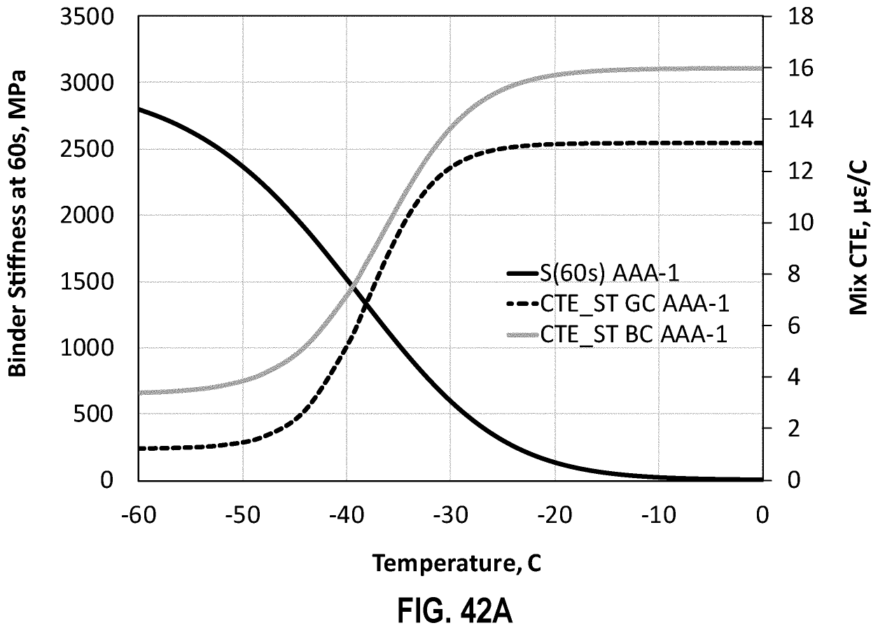

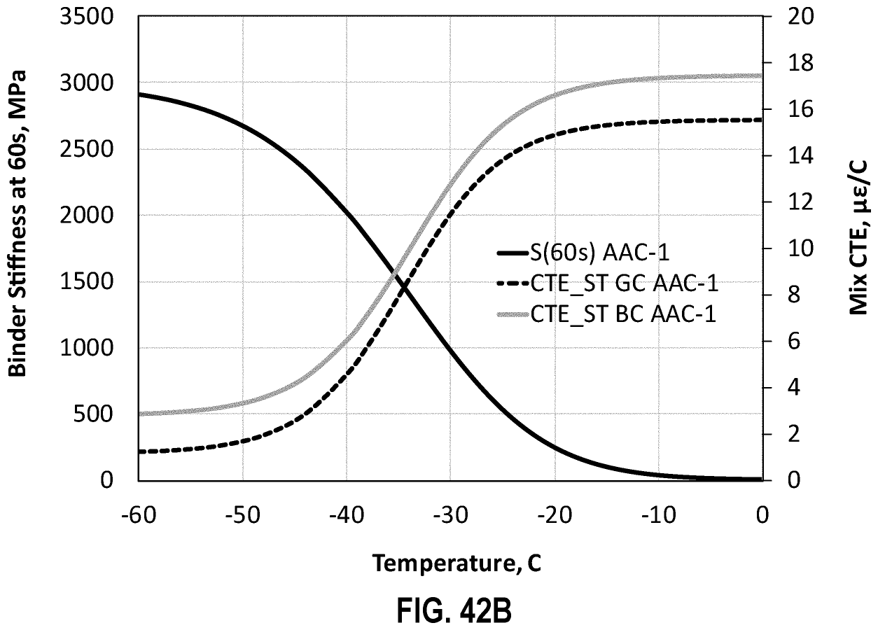

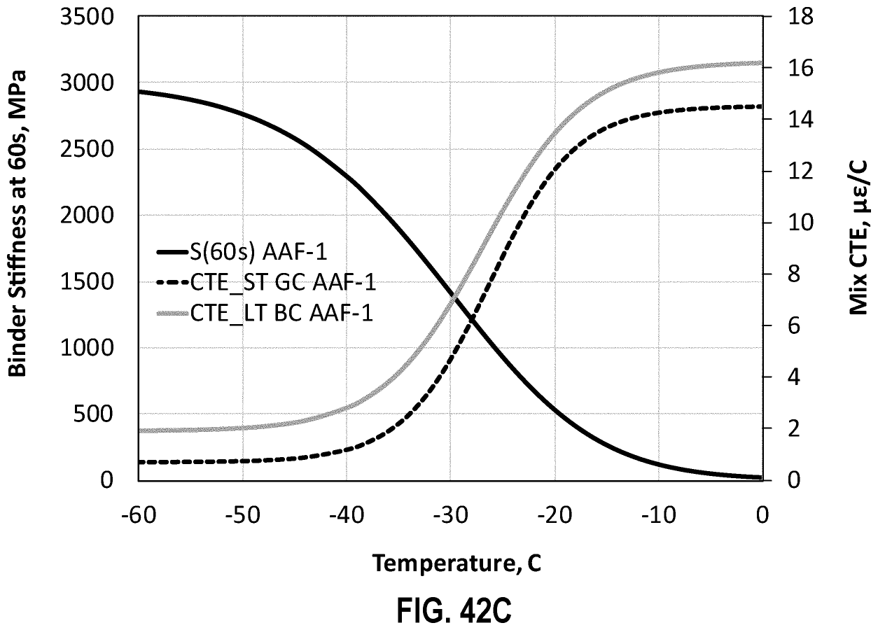

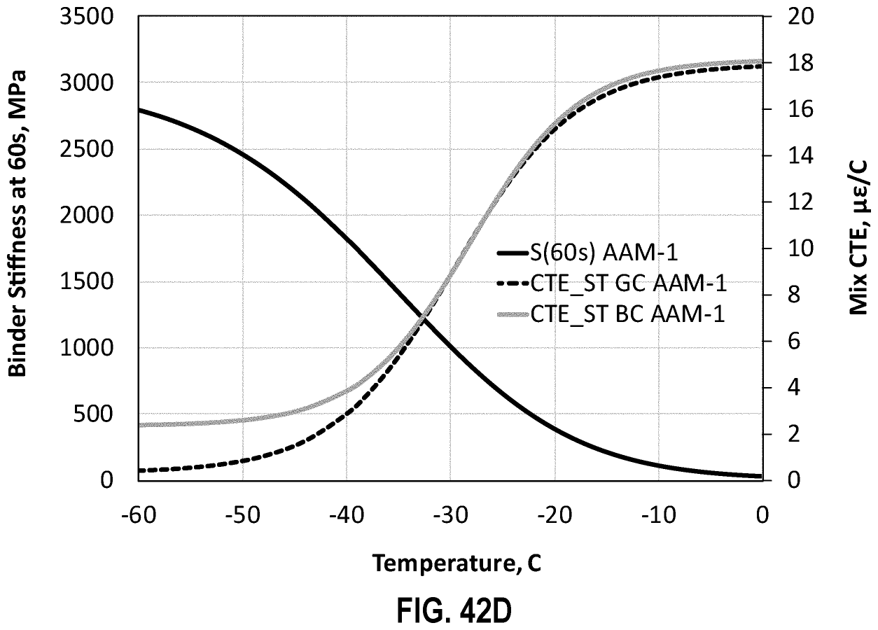

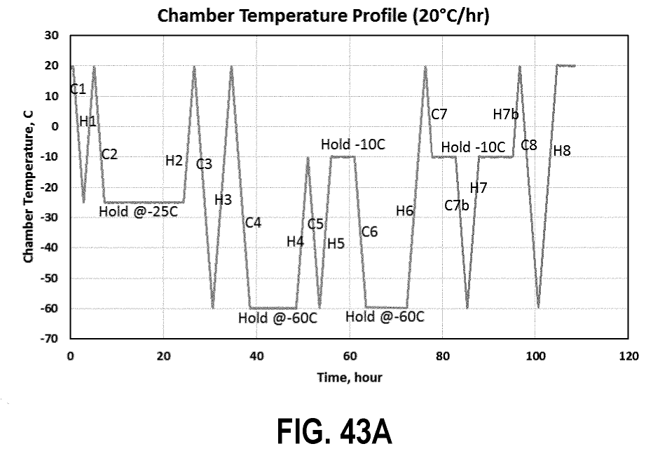

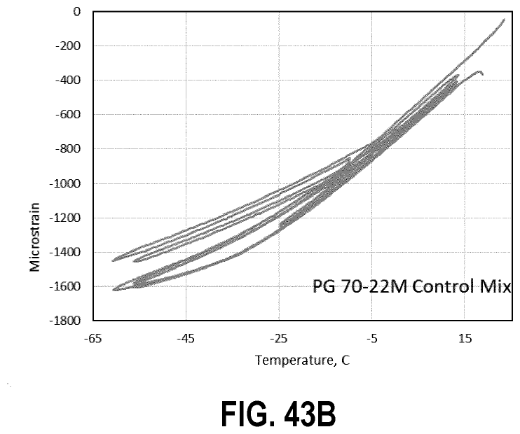

The CTE device test may be performed over a wide range of temperatures relevant to the low temperature thermal cracking of asphalt pavements. For example, in one embodiment, the CTE device test may be performed over a range of temperatures from +20° C. to −60° C. During a CTE device test, specimen deformations are measured using linear variable differential transducers and temperature detectors. For example, in the CTE device shown in FIG. 1, two linear variable differential transducers (LVDTs) are placed substantially perpendicular to each other. In addition, chamber temperature, CTE device frame temperature, sample surface temperature, and sample interior temperature can be measured using four resistance temperature detectors (RTDs). In one embodiment, measurements may be taken every 60 seconds (such measurements can be seen in FIG. 39).

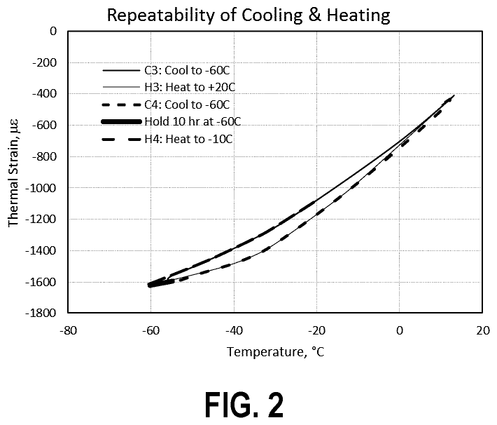

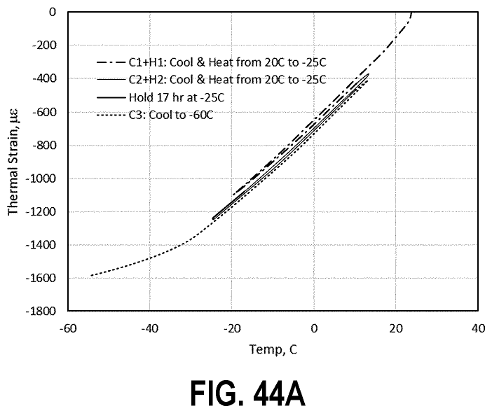

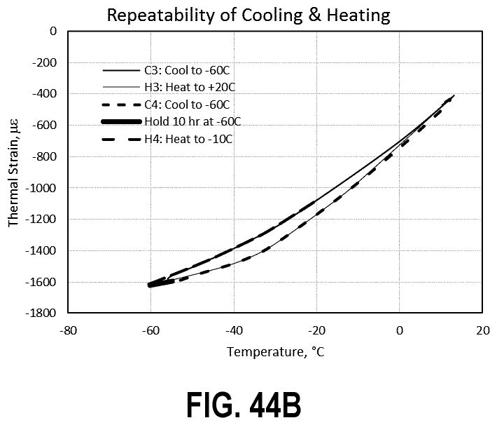

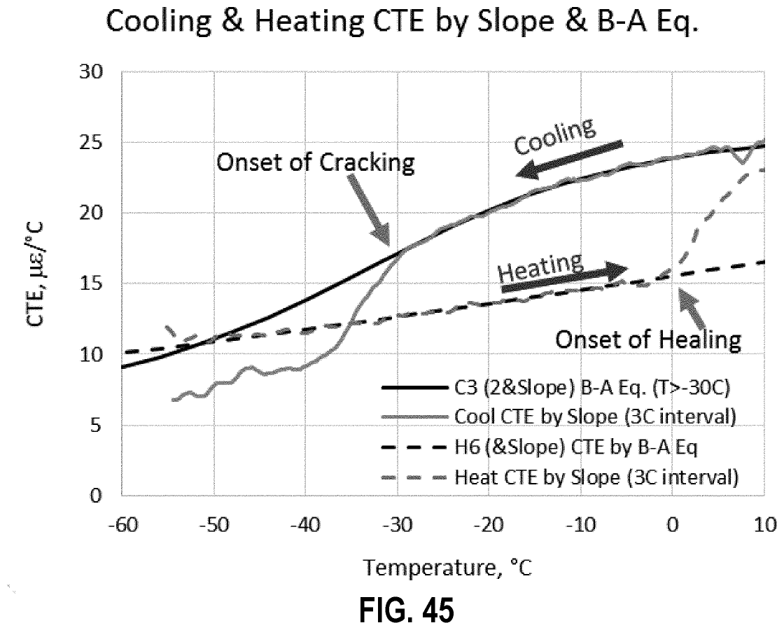

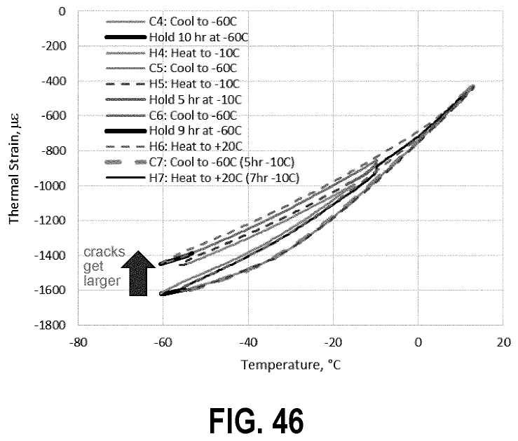

The CTE device can be used to determine CTE of various asphalt mixtures. In one method of use, the CTE device measures the dimensional changes of test specimen in two mutually perpendicular diametric directions as the temperature is lowered or raised at a predetermined rate (typically 10° C./hour or 20° C./hour). A typical CTE device measurement (based on such a method) is shown in FIG. 2, which shows thermal strain versus temperature plot. In the figure, C3, H3, C4, and H4 represent cooling and heating cycles that the sample underwent. This data represents the third and fourth cycles (of seven total cycles). The data for all cycles can be seen in FIGS. 44A, 44B, and 46, and will be discussed in greater detail below. The linear CTE value is the slope of the curve and the test results were very much repeatable; this can be seen as two consecutive tests closely overlap one another.

The CTE values of all asphalt mixture tested with the CTE device vary with temperature and show a distinct transition at a very low temperature, usually between −40° C. to −20° C. Further, the measured CTE were different for cooling and heating. Up until the work of the present inventor, researchers had believed that the transition is the result of phase change from a liquid state at warm temperatures to a glassy state at low temperatures (consequently called a glass transition temperature—T_{g}); however, researchers had no explanation for the different CTE values for cooling and heating cycles.

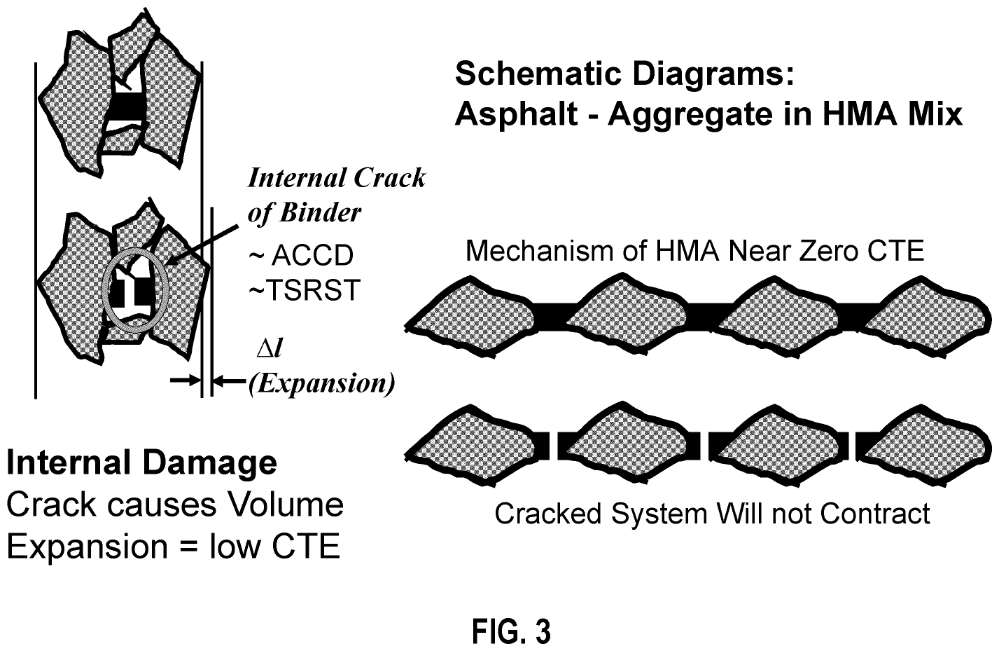

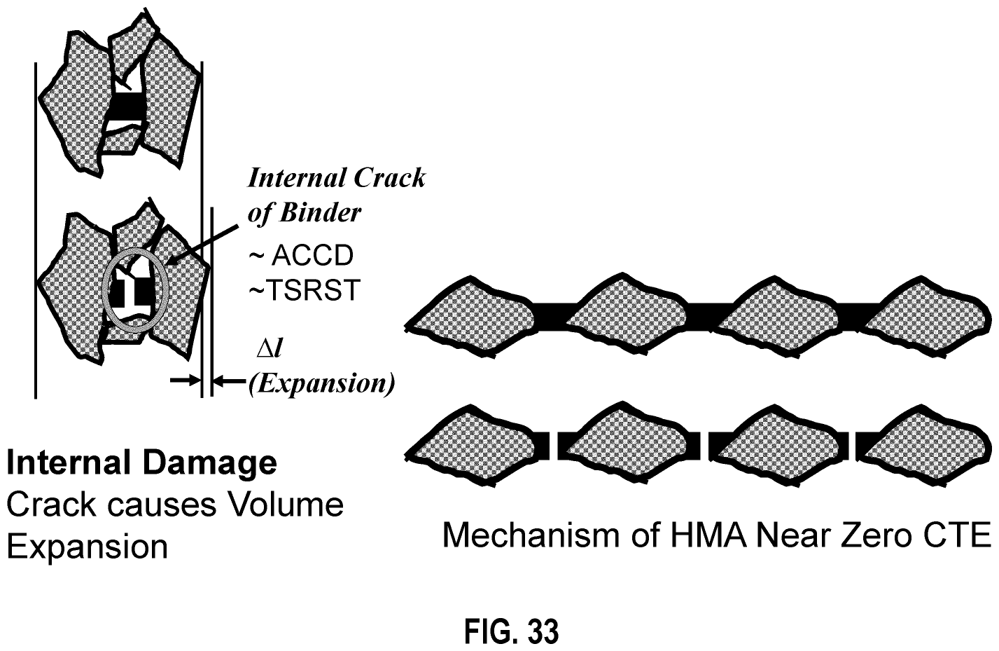

The work of the present inventor described herein, however, determined that the transitions were the result of internal cracking induced by differential thermal contraction of asphalt binder and aggregate—and the present inventor also determined that this can be used for prediction of the low temperature thermal cracking of asphalt. Furthermore, the work of the present inventor determined that the different CTEs for cooling and heating were the phenomena caused by the development of internal cracks. The mechanism of internal damage of asphalt mixture at low temperatures is believed to be due to the differential thermal contractions. Asphalt mixture is a composite consisting of asphalt binder and mineral aggregates, where asphalt and aggregates are bonded together as shown in FIG. 3. While the CTE values for aggregates range between 5 με/° C. to 15 με/° C., the CTE values for asphalt binders are much larger with typical values ranging between 15 με/° C. to 300 με/° C. As temperature is lowered from an ambient temperature to a low temperature, asphalt binder placed between aggregates contracts more rapidly than the bonded aggregates, developing a tensile stress within the asphalt binder and pulling asphalt mixture inward (contraction). As the temperature continues to drop, the tensile stress within the asphalt binder continues to increase. Ultimately, asphalt binder starts to fracture at locations where the tensile stress reaches the failure strength. As the fracturing of asphalt binder continues at more locations, the inward stress that made the mixture contract is gradually reduced and some portion of already developed contraction recovers (expand), but very slowly due to high stiffness of asphalt binder at low temperatures. This new model developed by the present inventor also explains the extremely low CTE values of asphalt mixtures measured by the CTE device at very low temperatures, near −60° C. As shown in FIG. 3, when sufficient level of asphalt binder cracking occurs, the whole asphalt mixture may experience very small contraction, causing a near-zero CTE (see also FIG. 33).

This proposed internal cracking mechanism developed by the present inventor was validated with three experiments.

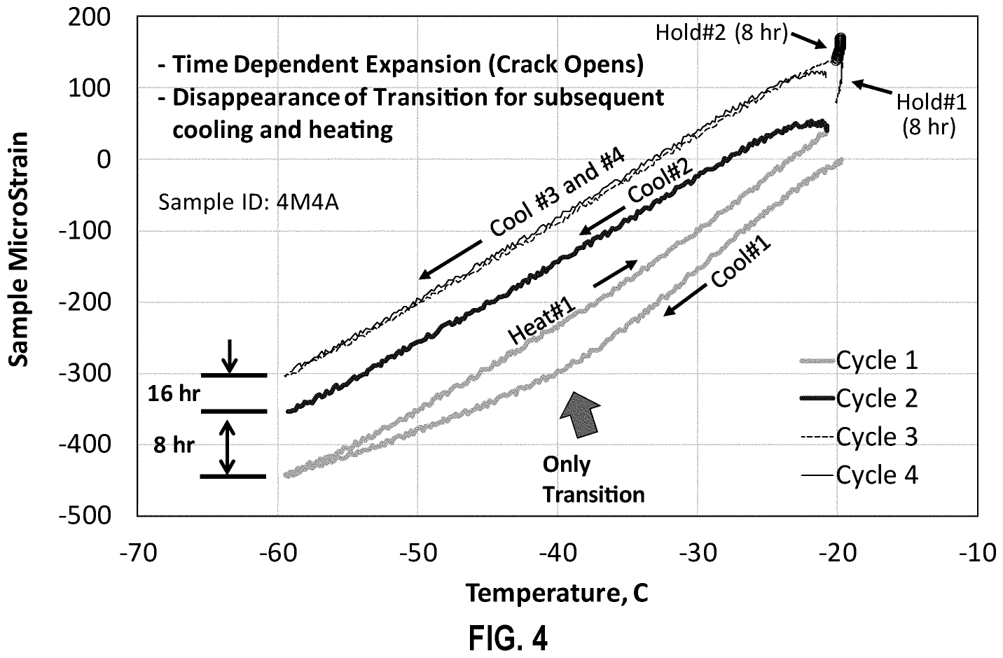

In the first validation experiment, an asphalt mixture sample was cooled to −60° C. and warmed up only to −20° C. and repeated cooling and heating between −60° C. and −20° C. for four times. The thermal strain versus temperature plot is presented in FIG. 4. On the first cooling cycle, the transition was observed near −38° C. On seven subsequent temperature cycles either cooling or heating cycles, no transition was observed. Unlike the CTE device test result shown in FIG. 2 where the sample was warmed up to 10° C. where the temperature is warm enough for healing of cracks, transition or internal thermal cracking happened for both temperature cycles. However, for test shown in FIG. 4, sample was warmed up only to −20° C. where the temperature is too low for healing to take place. After the first cooling and transition (cracking), the thermal contraction of sample recovered and the sample strain slowly increased over about 24 hour period.

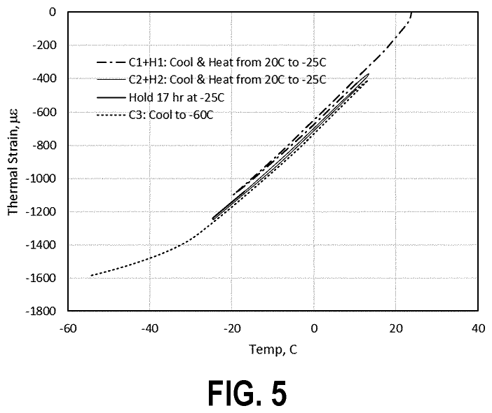

In the second validation experiment as presented in FIG. 5, an asphalt mixture sample was cooled from an ambient temperature only to −25° C., warmer than the expected transition (or cracking) temperature, then subjected to −25° C. to 20° C. temperature cycles. For the last cooling cycle, the temperature was lowered to −60° C. to determine the transition temperature and it was approximately −35° C. For −25° C. to 20° C. temperature range, there is almost no difference between cooling CTE and heating CTE. This experiment suggests that when asphalt mixture is cooled below the transition temperature, many cracks (internal thermal cracks) develop within the sample, altering thermal response causing different CTEs for cooling (undamaged condition until the transition temperature) and heating (damaged condition throughout). The gradual increase of thermal strain (curve moves downward) with temperature cycle are believed to be creep strain caused by self-weight.

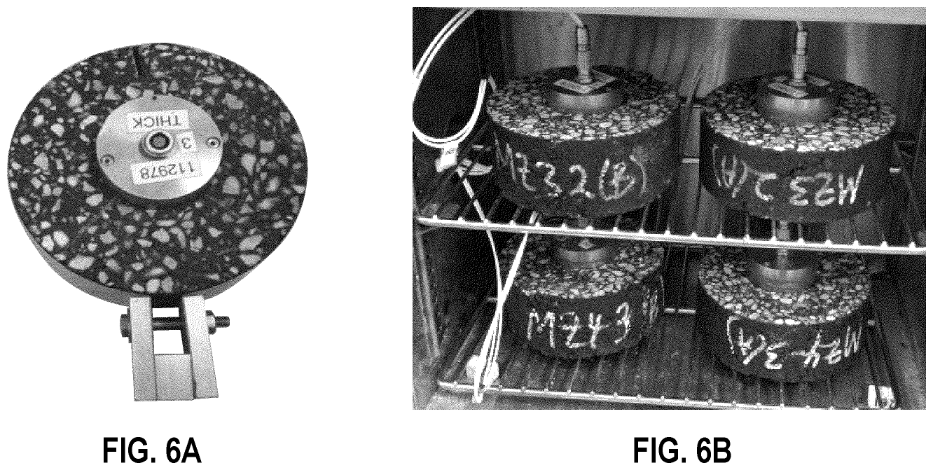

The third validation experiment was a comparison of the CTE device transition temperatures with cracking temperatures measured by existing asphalt mixture test methods. For this experiment, eight asphalt mixtures were prepared with four asphalt binders and two aggregate sources. The eight mixtures were tested with the CTE device and an asphalt concrete cracking device (ACCD). ACCD measures the thermal cracking temperature by creating a field-like condition and detect cracking of the test specimen. As shown in FIGS. 6A and 6B, 150 mm diameter and about 110 mm tall asphalt sample was cut in half and then a core was removed from the middle, and the void of the core fitted with an ACCD ring, which has near zero CTE. As temperature drops, asphalt sample contracts and the ACCD prevents the contraction, inducing tensile stress within the sample. Continued cooling will cause eventual failure at a notch. Each ACCD ring is instrumented to measure temperature and strain.

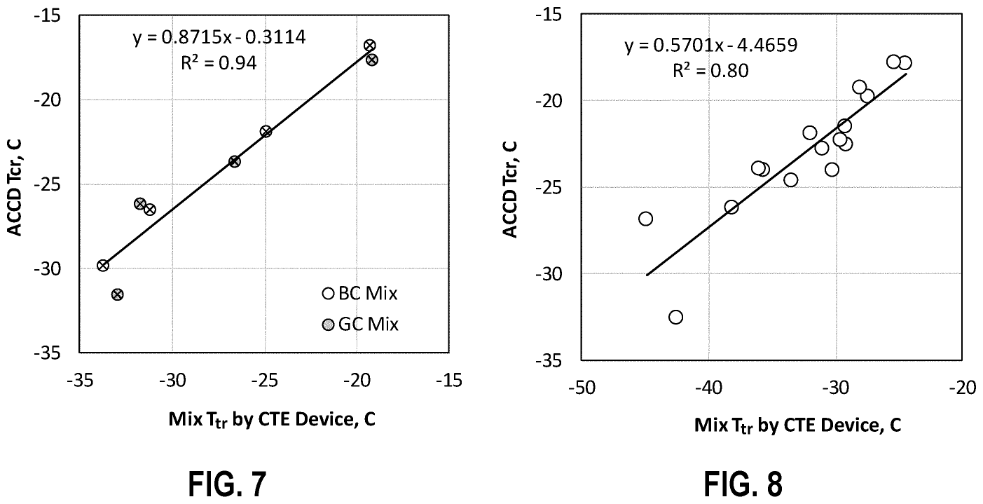

Based on data from these tests, the CTE device transition temperature is highly correlated with the mixture thermal cracking temperature measured by ACCD as shown in FIG. 7.

ACCD cracking temperatures and CTE device transition temperatures were also measured for various asphalt mixture types, including hot mix, warm mix, polyphosphoric acid modified, polymer modified, recycled asphalt pavement, recycled asphalt shingle. A comparison between ACCD and CTE device results is shown in FIG. 8 [as performed per Kim, S., M. Nazzal, A. Abbas, M. Akentuna, and M. Arefin, “Evaluation of Low Temperature Cracking Resistance of WMA,” Final Report FHWA/OH-2015111 (2015)].

For CTE device measurement in the study, not knowing the importance of the transition temperature, the temperature was lowered only to −40° C. chamber temperature (about −35° C. sample temperature), causing bigger variability in the CTE device transition temperature and little weaker correlation than the study where temperature was lowered to −60° C.

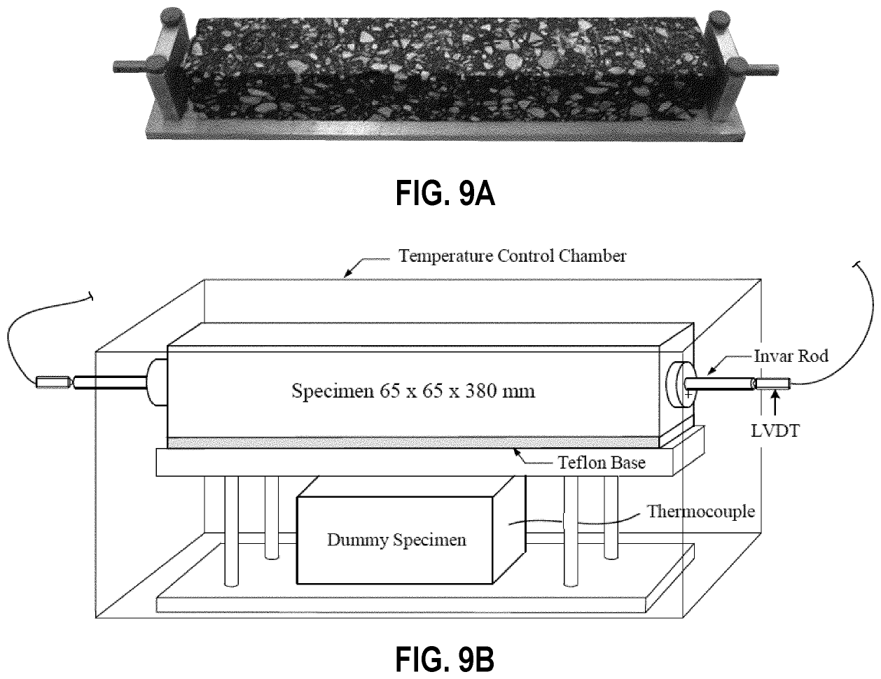

Marasteanu et al., (2007) conducted extensive study on low temperature cracking of asphalt pavement where they measured CTEs and the thermal cracking temperatures for various mixtures. For CTE and the transition temperature (or glass transition temperature as they called), they used Asphalt Thermal Cracking Analyzer (ATCA) as shown in FIGS. 9A and 9B, where an asphalt beam sample was cooled by liquid nitrogen at a rate of 60° C./hour and the dimensional change was measured by two LVDTs.

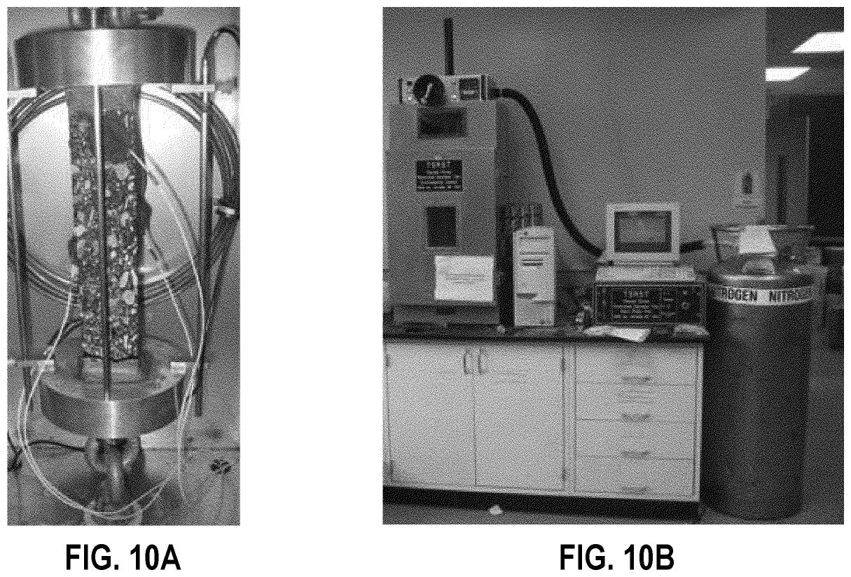

For thermal cracking temperature, Marasteanu et al. used the Thermal Stress Restrained Specimen Test (TSRST) as shown in FIG. 10 where an asphalt beam was glued to two metal platens at both ends and subjected to a constant cooling (10° C./hour). During the test, the thermal contraction was restrained by applying tensile force to keep the sample length constant throughout the test. As temperature decreases, the thermal tensile stress within the beam increases. The temperature at which the specimen fracture is TSRST cracking temperature (T_{cr}).

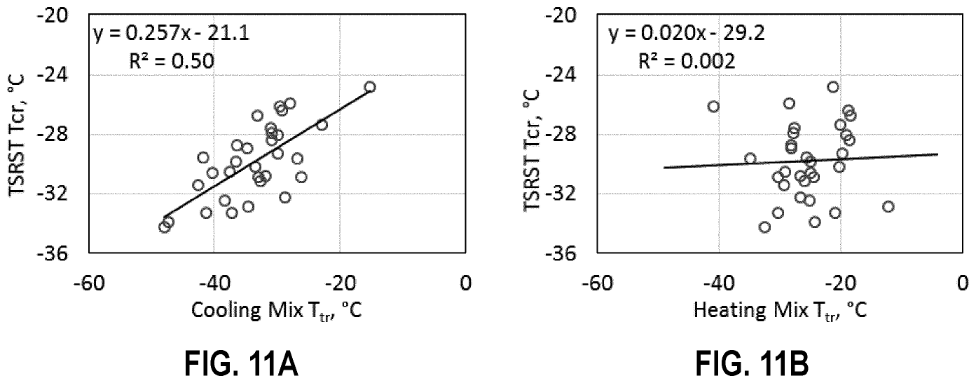

The TSRST cracking temperature and CTE transition temperature data listed in their report (Marasteanu et al., 2007) are extracted and plotted in FIG. 11. The cooling CTE transition temperature shows a statistically significant correlation with the TSRST cracking temperature most likely because the CTE transition is the result of thermal contraction of test specimen as in TSRST. However, the heating CTE transition temperature does not show any trend with the TSRST cracking temperature because there will not be a thermal cracking process in heating CTE measurement. The weaker correlation (or lower R², coefficient of determination) is probably due to poorer repeatability of TSRST than ACCD, and smaller number of specimens for each mixture (2 for TSRST versus 4 for ACCD). In addition, ATCA uses 60° C./hour cooling rate, creating a large temperature gradient between specimen surface and the middle and more test variability than the CTE device test with 20° C./hour.

Thus, the CTE device described herein, is a good fit to the present needs of industry because of its simplicity, repeatability, and accuracy.

In that regard, the CTE device can measure low temperature cracking potential and CTE easily, precisely, and accurately using thin slice of cylindrical specimen. As described, the CTE device frame (and test specimen) is placed inside of a microprocessor-controlled environmental chamber that can cool the chamber contents to −60° C. or colder in a well-controlled manner. Dimensional changes and temperatures are measured by two LVDTs and four RTDs, respectively. LVDTs are placed mutually perpendicular diametric directions and RTDs are placed to measure temperatures of chamber, CTE device frame, surface and interior of the test specimen.

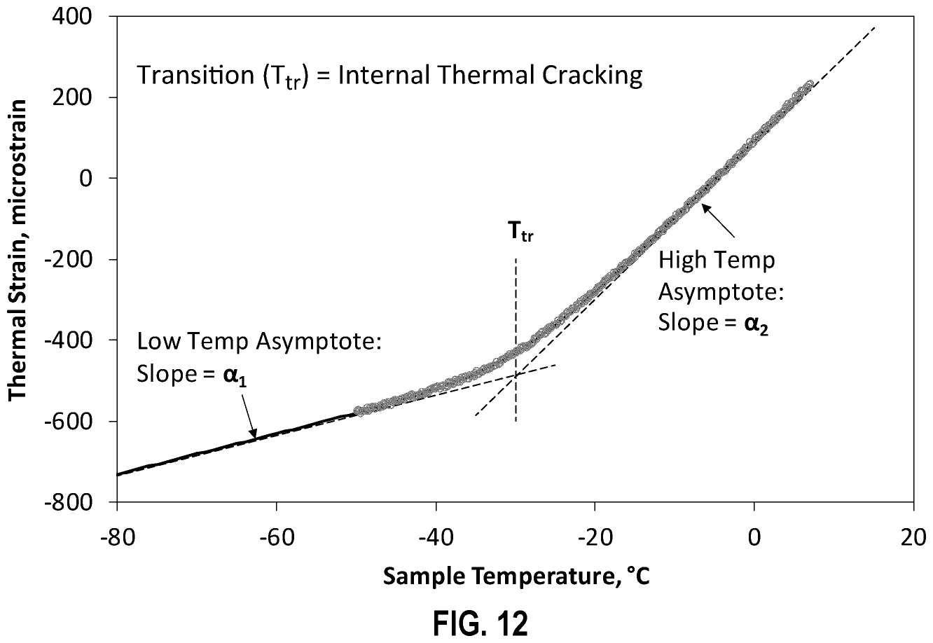

One can then calculate corrected deformation and strain for each specimen and temperature—accounting for frame contraction of the CTE device frame with frame temperature. Thermal strain versus average specimen temperature can be plotted and determine the slope (CTE) and the transition temperature using an appropriate analysis procedure as shown in FIG. 12. The differential thermal contraction of asphalt binder and aggregate causes the transition, and the transition temperature (Tₜᵣ) is the measure of the low temperature cracking potential of the asphalt mixture.

And so, one aspect of the present invention is directed to a method of predicting or determining temperature for thermal cracking of a composite material. Such method includes: (1) reducing the temperature of a composite material along a range of temperatures from a first temperature to a second temperature; (2) measuring dimensional changes in the composite material at a plurality of temperature points along the range of temperatures to generate a curve related to values for the coefficient of thermal expansion for the composite material; and (3) determining the transition temperature for the composite material. The transition temperature may be determined as being at the intersection of two asymptotes of the curve, wherein the transition temperature correlates to the thermal cracking temperature of the composite material.

In this aspect of the present invention, the composite material may be an asphalt mixture. Further, the first temperature may be about 20° C. and the second temperature may be about −60° C. Reducing the temperature of the composite material may be performed at a rate chosen from 10° C. per hour and 20° C. per hour. And, measuring dimensional changes in the composite material may further include obtaining a first measurement of dimensional change and a second measurement of dimensional change. Additionally, the first measurement of dimensional change and the second measurement of dimensional change may be taken in a manner substantially perpendicular to one another. In certain embodiments, the first measurement and the second measurement may be taken by a first linear variable differential transducer and a second linear variable differential transducer.

The method of this aspect of the present invention may further include obtaining a plurality of temperature measurements of the sample. Such plurality of temperature measurements of the sample may include a first measurement taken at an exterior surface of the sample, and a second measurement taken at the interior of the sample. Additionally, the plurality of temperature measurements further includes a third measurement taken of an interior space of a chamber, wherein the composite material is positioned in the interior space. The positioning of the composite material within the interior space may occur within a frame disposed within the interior space. Further still, the plurality of temperature measurements may further include a fourth measurement taken of the frame. The plurality of temperature measurements may each be taken at sixty second intervals.

Some other aspects of the present invention include: (1) diametric measurement allows two measurements on single specimen; (2) V-shape bottom of the test frame allow self-alignment of sample set-up, reducing operator time on test; (3) the CTE device test frame was designed to use waste asphalt samples already used for routine density measurement for common QC test and then recycled (and so, no efforts are needed for preparing samples; it only requires slicing the sample into two pieces to reduce the thickness and minimize temperature gradient within the sample); (4) by using internal thermal cracking, contribution of all mixture components are evaluated in similar manner as in the field conditions; (5) the CTE device can accurately measure CTE of asphalt mixture and Portland cement concrete in dry or moisture saturated condition; (6) freeze-expansion of sample measured by the CTE device may be related to the degree of damage; and (7) the CTE device may be able to measure high temperature (50° C.) creep caused by self-weight (high temperature creep is a most common property measured to prevent rutting problems).

### EXAMPLE

Introduction

This example presents a more detailed description of the experiments described above to validate the present inventor's model of internal cracking in composite materials (such as asphalt mixtures), and presents further data and discussion that results in the present inventor's new model of CTE for prediction of low temperature cracking of composite materials (such as asphalt mixtures). This example was performed by the present inventor and studied factors affecting CTE and low temperature cracking performance of asphalt mixtures, and thermo-volumetric behaviors of asphalt mixtures at low temperatures.

As described above, major factors in low-temperature cracking of asphalt mixtures include stiffness, CTE, and strength. A representation of this low temperature cracking criteria (for a single event) may be given as:

σₜₕₑᵣₘₐₗ=f {E(T,t), CTE(T)}≥σ_{strength}

As described above, however, current practice in determining or predicting low temperature cracking involves an analysis of mainly stiffness [BBR stiffness and m-value (i.e., slope of the master stiffness curve at 60 seconds)]—without incorporating other factors (including CTE) in that analysis. The challenge in studying CTE is that there are many other factors that also affect E (stiffness), CTE and/or strength simultaneously. As a result (as discussed above), current practices are not adequate.

And so, the objectives of the studies of the present inventor (described in this Example) were to: (1) determine factors affecting the coefficient of thermal expansion/contraction (CTE) of asphalt mixtures; (2) determine (pure) CTE effects on low temperature cracking temperature by using an Asphalt Concrete Cracking Device (ACCD); (3) study and understand roles of mixture components on thermo-volumetric behavior of asphalt mixture; and (4) analyze damage that can be caused by moisture (salt) freezing and thawing.

Device and Method

For the experiments performed in this Example, a CTE device as described above was used. The device (as shown in FIG. 1) includes an aluminum frame. Two linear variable differential transducers (LVDTs) are spaced 90° apart. The device received a gyratory specimen that had been cut to be 50-55 mm thick. The specimen was conditioned at 20° C. for 30min. It was then cooled to −60° C. (or −40° C.) at a rate of 20° C./hr. The device further includes 4 resistance temperature detectors (“RTDs”), which are placed on (1) the surface of the sample, (2) the middle of the sample, and (3) at two locations on the device frame. Data collection occurred every 60 seconds for the data discussed in this Example. Calibration was performed with Invar and Titanium Silicate, and the experimental runs validated with AL 6061 (aluminum alloy) and SS 316 (stainless steel). As will be demonstrated below, the CTE device as described and used can provide data including, and can allow for an analysis of, CTE, low temperature performance, and a better understanding of a moisture damage mechanism.

Initial Experiments and Results

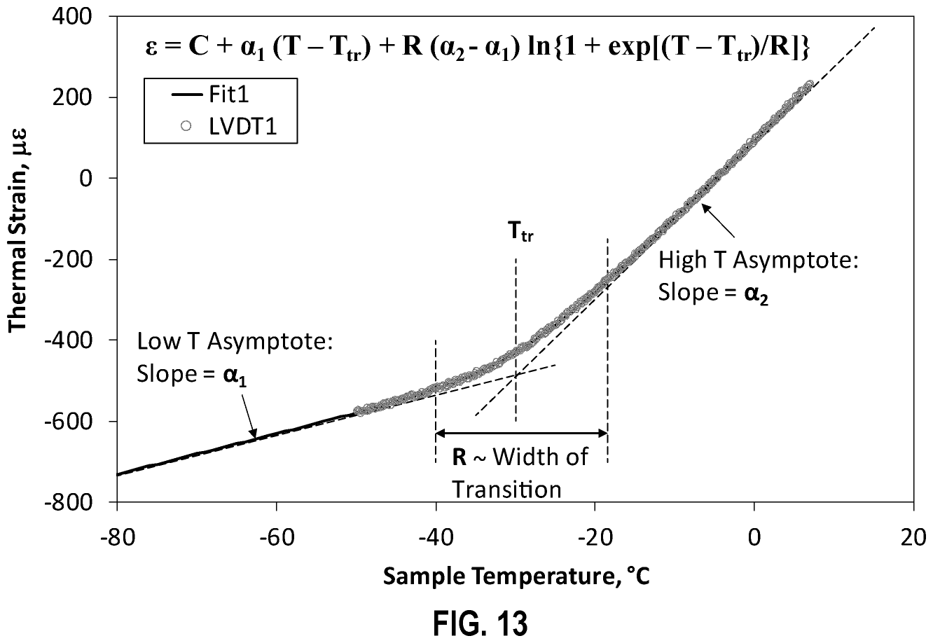

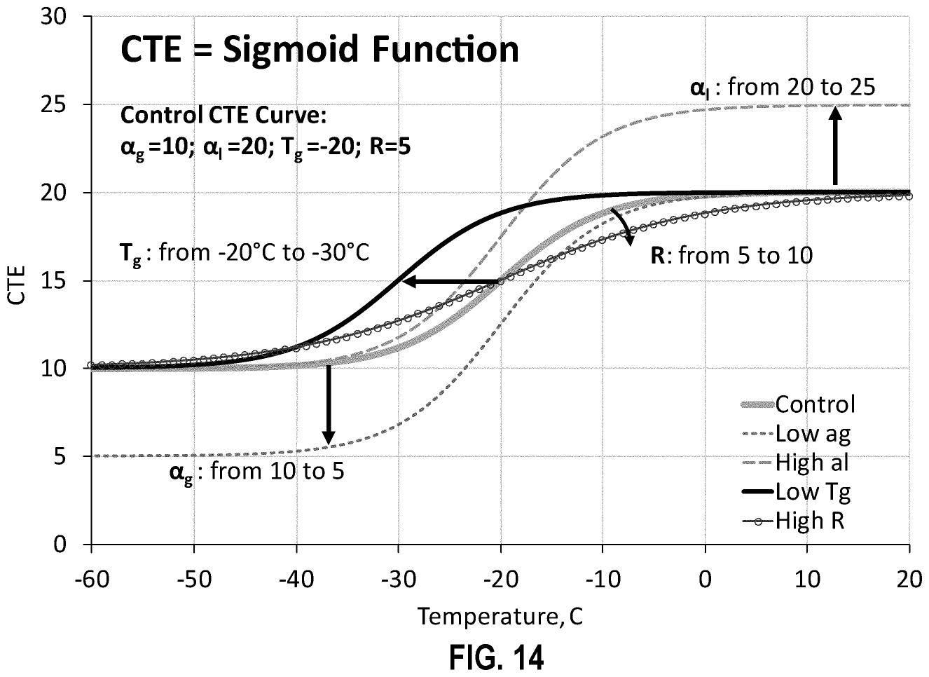

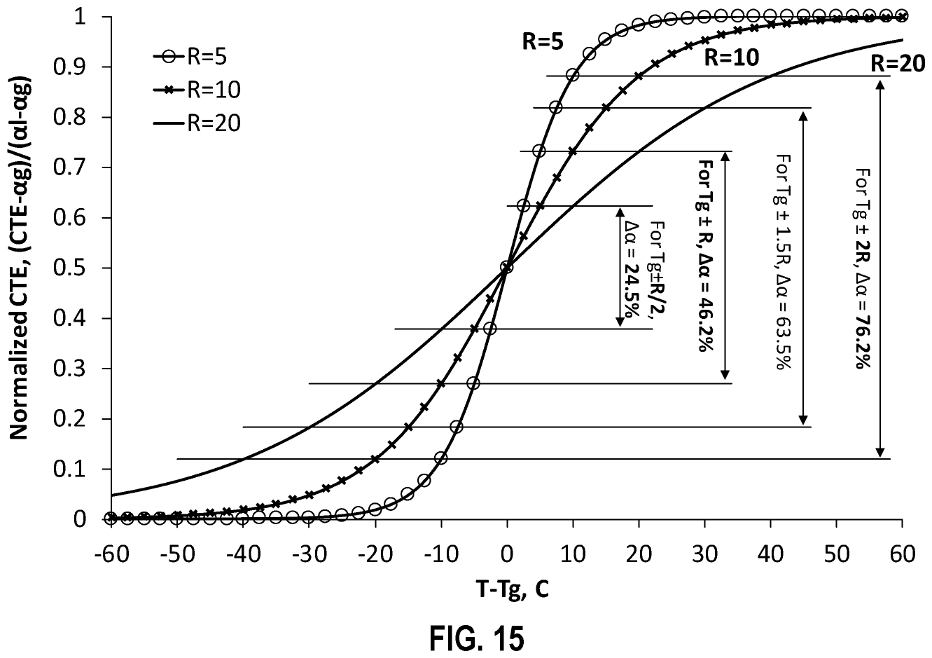

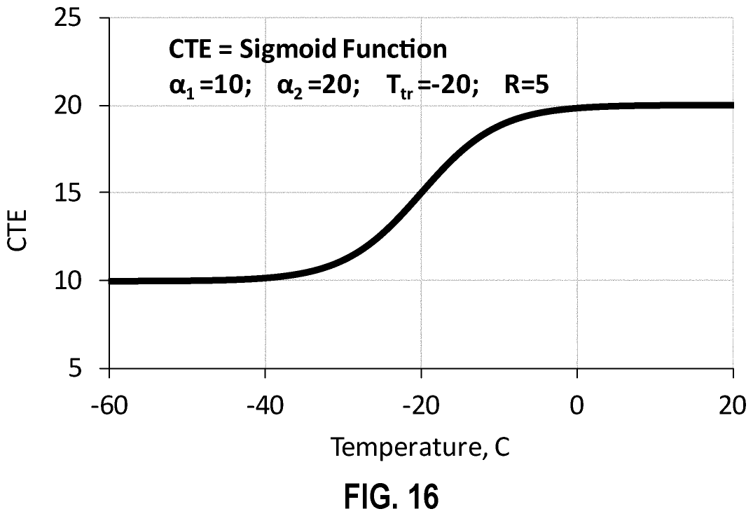

Initial results from tests of asphalt samples using the CTE device are shown in FIGS. 13-15. A plot of thermal strain data from the CTE device is shown in FIG. 13. Plotting data shows CTE as being a sigmoid function of temperature, as shown in FIG. 14. In FIG. 14, CTE=dε/dT, which equals α1 +(α2−α1)/[1+exp(-(T−Tₜᵣ)/R)]. And FIG. 15 is directed to the CTE transition rate. Following the running of samples on the device and obtaining the data, it was determined that the CTE device developed by the present inventor produces repeatable results. The standard deviation of CTE at single measurement was less than 0.3 με/° C.

Tests were then also run using an Asphalt Concrete Cracking Device (ACCD). And factors affecting CTE and factors affecting low temperature performance [as determined using the ACCD] were similar to that generated when using the CTE device. Binder grade, reclaimed asphalt pavement (RAP)/reclaimed asphalt shingles (RAS), aggregate, aggregate size, and aging were factors in both CTE and low temperature performance. Compaction (air voids) affected low temperature performance, but not CTE. Binder content affected CTE, but not low temperature performance.

The present inventor then also studied pure (or near-pure) CTE effects on low temperature performance. To do this, two aggregates (one having low CTE and one having high CTE) were selected from Ohio limestones. The selected aggregates were: (1) Grove City (GC) Limestone, having a CTE of 4.2 με/° C., and (2) Belle Center (BC) Limestone, having a CTE of 8.4 με/° C. Each of these aggregates was combined with natural sand in a aggregate to sand ratio of 85/15. This provided a CTE (for the combined aggregate and sand) of: 5.5 με/° C. for GC Limestone+natural sand, and 9.1 με/° C. for BC Limestone+natural sand.

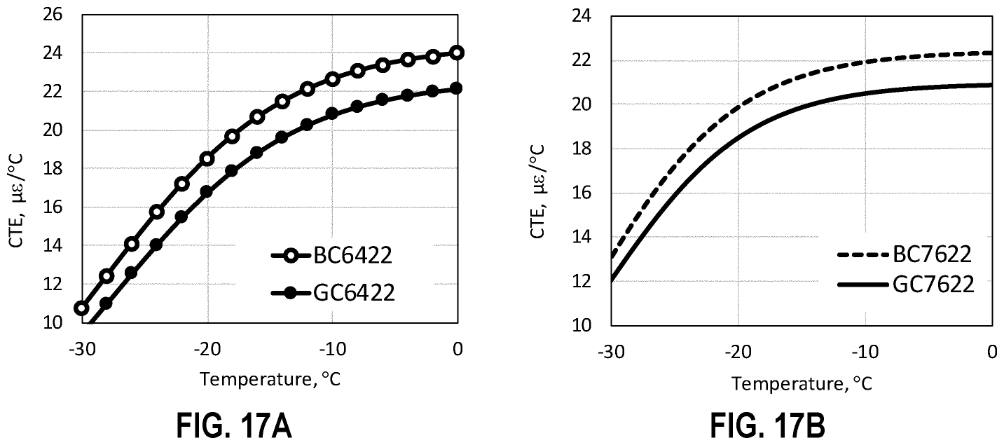

The aggregates were then sieved and recombined to the same gradation (Ohio DOT 12.5mm Superpave mixture). Two binders [PG 64-22 and PG 76-22 (SBS modified)] were used in the final mixture. This resulted in the testing of four mixtures [(1) GC aggregate +natural sand +PG64-22 binder (“GC6422”), (2) BC aggregate +natural sand +PG64-22 binder (“BC6422”), (3) GC aggregate+natural sand+PG76-22 binder (“GC7622”), and (4) BC aggregate +natural sand +PG76-22 binder (“BC7622”)]. Results showing the CTE effects on cracking temperature (T_{cr}) as measured with an ACCD are shown in FIGS. 17A and B (FIG. 17A showing the 6422 mixtures and FIG. 17B showing the 7622 mixtures). The BC6422 mixture had a T_{cr} of −19.2° C. The GC6422 mixture had a T_{cr} of −19.7° C. The BC7622 mixture had a T_{cr} of −21.9° C. And the GC7622 mixture had a T_{cr} of −24.0° C. This showed a statistically significant difference in CTE (p=0.000) and ACCD cracking temperature (p=0.004).

Next, the present inventor examined possible CTE contribution in cracking temperature (a theoretical calculation). For this calculation, strength and relaxation modulus (E) was assumed, and the present inventor then numerically solved for σ(t) as follows:

$$σ ( t ) = {\int }_{0}^{t} E ( ξ - {ξ}^{′} ) \frac{d ɛ ( τ )}{d τ} d τ$$

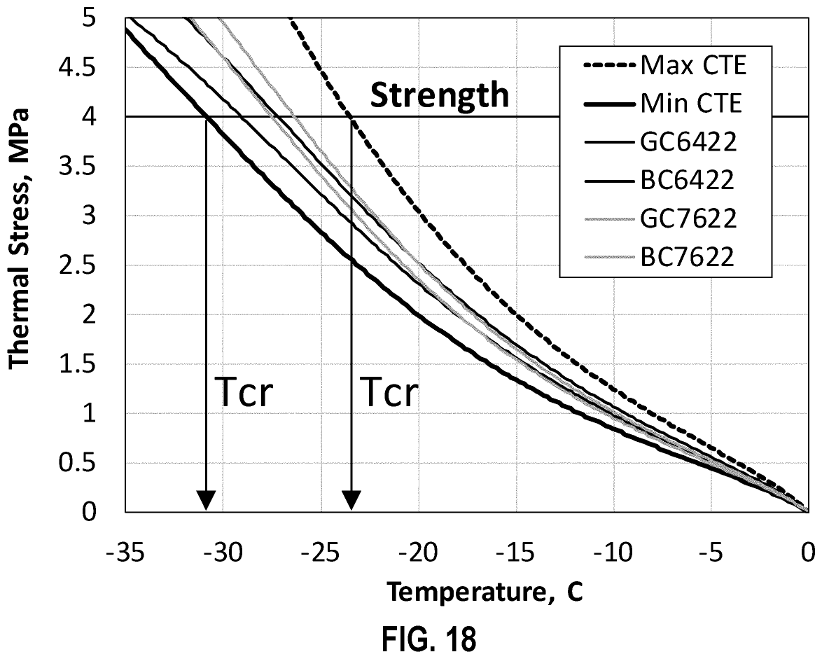

Results are shown in FIG. 18. The maximum and minimum CTEs out of the mixtures tested equaled a 7° C. difference in cracking temperature. This demonstrated the importance of CTE. (In ACCD tests, PG76-22 mixtures cracked at lower temperatures than PG64-22 due to partly higher strength).

Expanded Experiments and Results to Develop CTE Model for Prediction of Low Temperature Thermal Cracking

Following the initial results described above, the present inventor engaged in an expanded study to develop a better model for CTE. A starting point equation for developing a better model and equation for prediction of low temperature cracking was that of Pavement ME:

$${α}_{mix} = \frac{{α}_{agg} \cdot {V}_{a g g} + {α}_{b} \cdot {V}_{b}}{{V}_{t o t}}$$

The materials used in this expanded study included two aggregates and four binders—resulting in a total of eight mixtures (each of the aggregates being combined with each of the four binders. The two aggregates were (as in the initial experiment above) (1) Grove City (GC) Limestone, having a CTE of 4.2 με/° C., and (2) Belle Center (BC) Limestone, having a CTE of 8.4 με/° C. Each of these aggregates was combined with natural sand in an aggregate to sand ratio of 85/15. This provided a CTE (for the combined aggregate and sand) of: 5.5 με/° C. for GC Limestone+natural sand, and 9.1 με/° C. for BC Limestone+natural sand. [The combined aggregate CTE was determined using a natural sand CTE of 13.0 με/° C.—from Mukhopadhyay, Neekhra, Zollinger (2007).] Four Strategic Highway Research Program (SHRP) binders were used to create the final mixtures, each of the four mixtures being combined with each of the two aggregates. The four binders were (1) AAA-1, (2) AAC-1, (3) AAF-1, and (4) AAM-1 [Binder CTE data was obtained from the Association of Asphalt Paving Technologists (AAPT) -1993]. The aggregates were sieved and recombined to the same gradation (Ohio DOT 12.5 mm Superpave mixture), with a 5.7% binder content. (Hereafter, and in the figures, each mixture or binder may be referred to either with AAA-1, AAC-1, AAF-1, AAM-1, or simply with A, C, F, or M.)

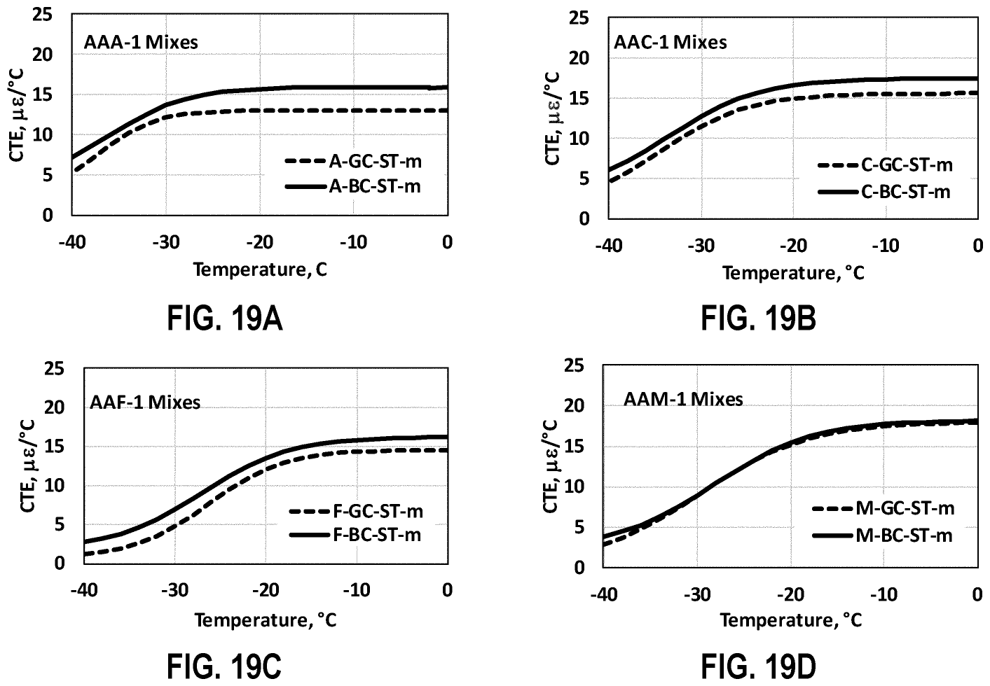

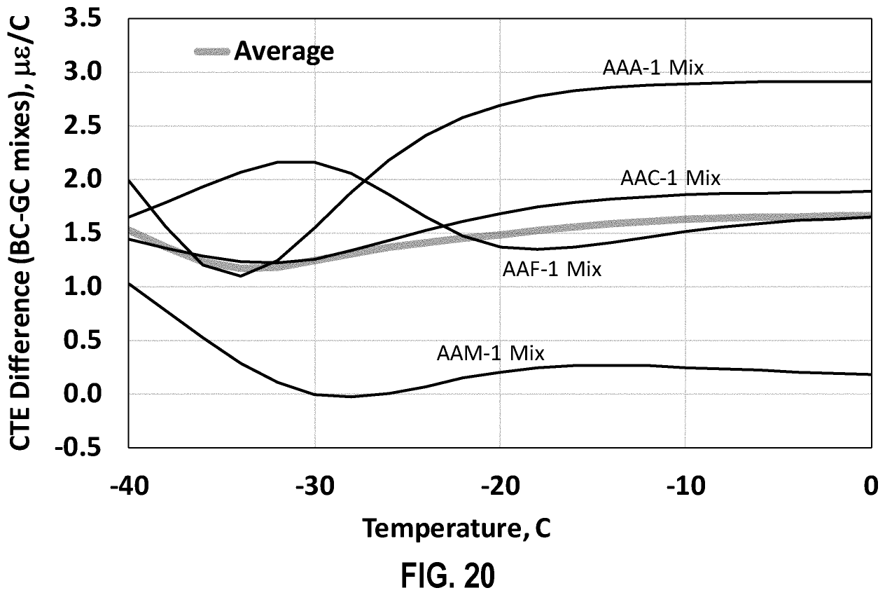

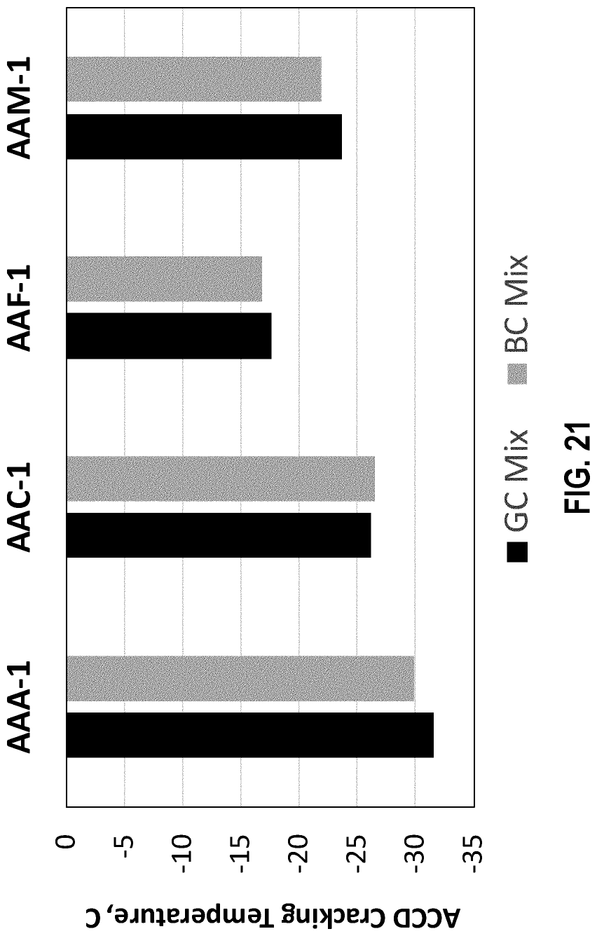

Each of the eight mixtures was then tested in the CTE device from 20° C. to −60° C. The aggregate effect (at 85.1% volume): 5.5 με/° C. vs 9.1 με/° C. (4.7 με/° C. vs 7.74 με/° C.—i.e., 4.7 με/° C. is 85.1% of 5.5 με/° C., and 7.74 με/° C. is 85.1% of 9.1 με/° C.). Results of CTE over that range of temperature are shown for each of the eight mixtures in FIGS. 19A-19D (mixtures including binder AAA-1 are in FIG. 19A; mixtures including AAC are in FIG. 19B; mixtures including binder AAF-1 are in FIG. 19C; and mixtures including binder AAM-1 are in FIG. 19D). FIG. 20 then shows the CTE difference across the range of temperature due to the aggregate (each BC mixture CTE at a particular temperature minus the corresponding GC mixture CTE at that particular temperature)—with the average difference also being shown. In making these measurements, the expected CTE difference was 3.0 με/° C. (calculated by the difference between 7.7 με/° C. and 4.7 με/° C.). However, as can be seen from FIG. 20, the average measured difference in CTE (based on aggregate used) was 1.50 με/° C. And, FIG. 21 is a graph depicting ACCD cracking temperature for each of the particular 8 mixtures prepared as described above.

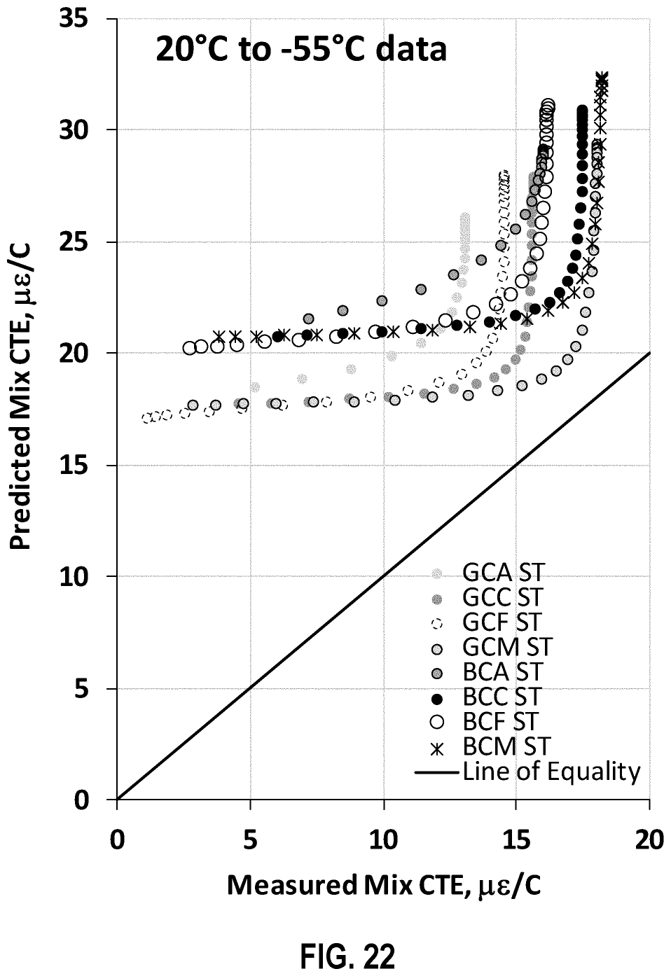

Next, the predicted CTE for each mixture was plotted against the measured CTE for each mixture. This plot is shown in FIG. 22.

$${α}_{m i x} ( T ) = \frac{{β}_{a g g} \cdot {V}_{a g g} + {β}_{b} ( T ) \cdot {V}_{b}}{3 \cdot {V}_{t o t}}$$

$$or$$

$${α}_{m i x} ( T ) = \frac{{α}_{a g g} \cdot {V}_{a g g} + {α}_{b} ( T ) \cdot {V}_{b}}{{V}_{t o t}}$$

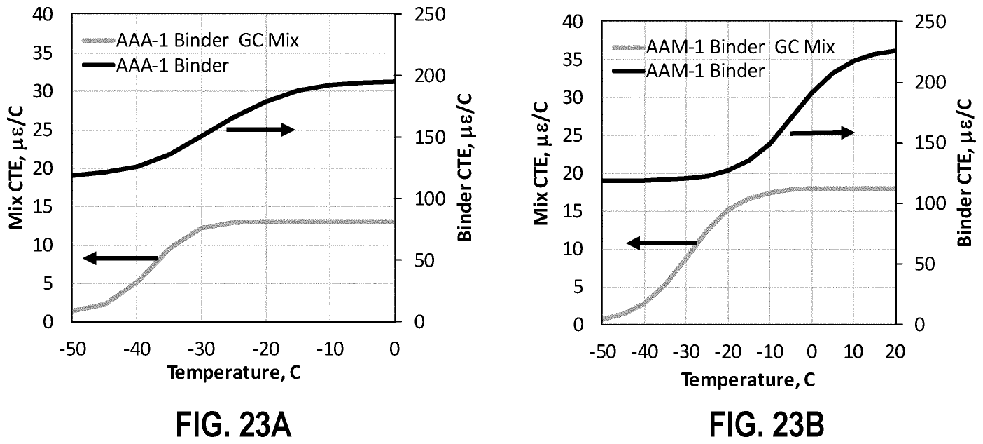

Others have reported a poor correlation between binder and mixture thermal properties. And so, FIGS. 23A and 23B depict graphs showing plots of binder CTE and mixture CTE for two of the binders (AAA-1 and AAM-1) and two mixtures [the two binders each mixed with Grove City (GC) Limestone mixture]. Binder CTE data was obtained from Bahia and Anderson AAPT (1993). As can be seen, binder glass transition and mixture transition do not match in either figure, suggesting different phenomena (or mechanisms) or that they may not be related.

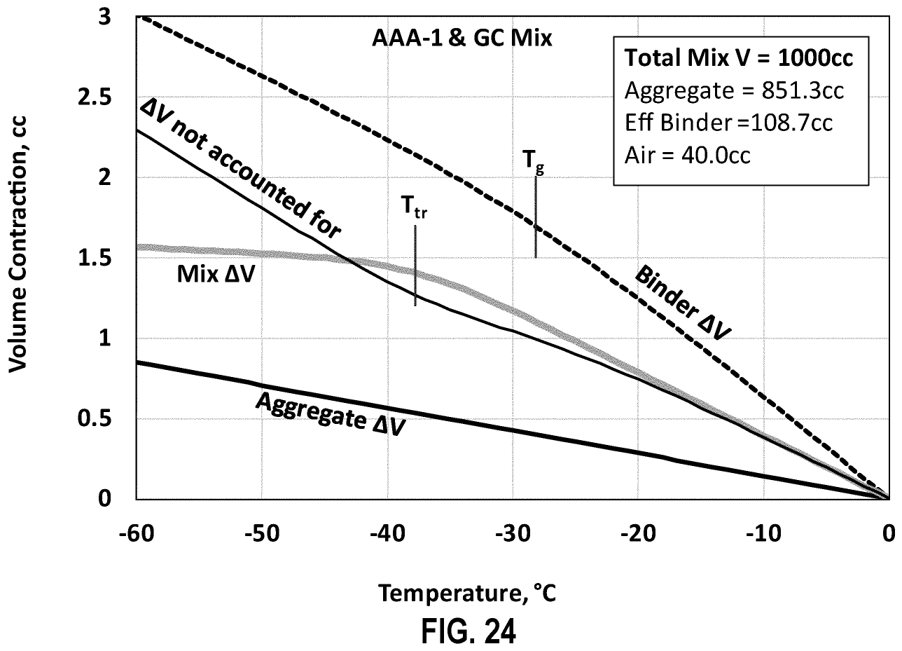

Next, the volume change over a range of temperature change (from 0° C. to −60° C.) for the various components of asphalt mixture was measured and plotted. The resulting graph is shown in FIG. 24. In the mixture measured (GC aggregate with AAA-1 binder), total mixture volume was 1000cc, the aggregate was 851.3cc, the binder was 108.7cc, and air was 40.0cc. β was assumed to be equal to 3α. The CTE equation used is:

$${α}_{m i x} ( T ) = \frac{{α}_{a g g} \cdot {V}_{a g g} + {α}_{b} ( T ) \cdot {V}_{b}}{{V}_{t o t}}$$

Conditions for the CTE equation to work would include a force equilibrium and constant relative distances among aggregates. The reality, however, is that there is uneven binder film thickness, which results in uneven stresses. And there is a local temperature gradient. As a result, the binder flows and fractures and the aggregates move.

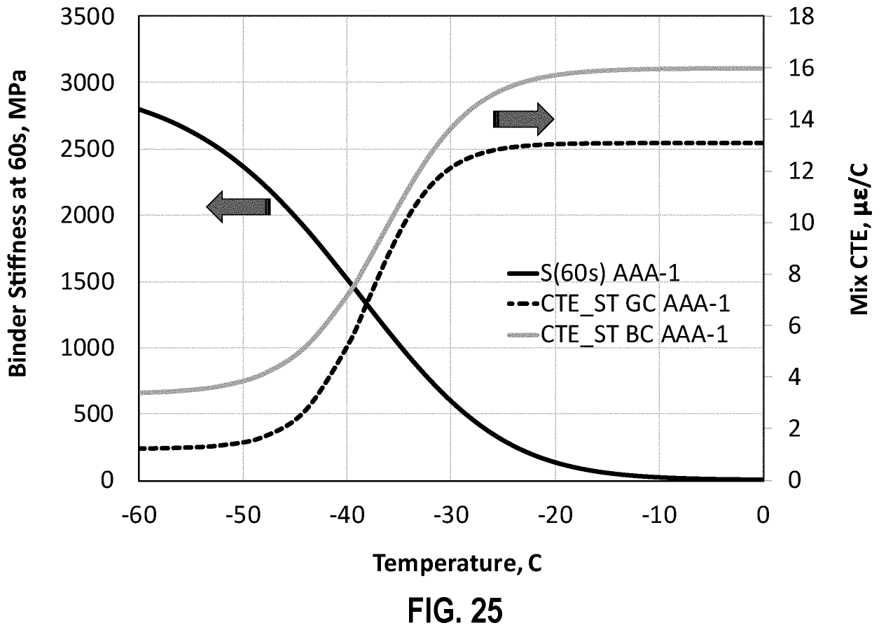

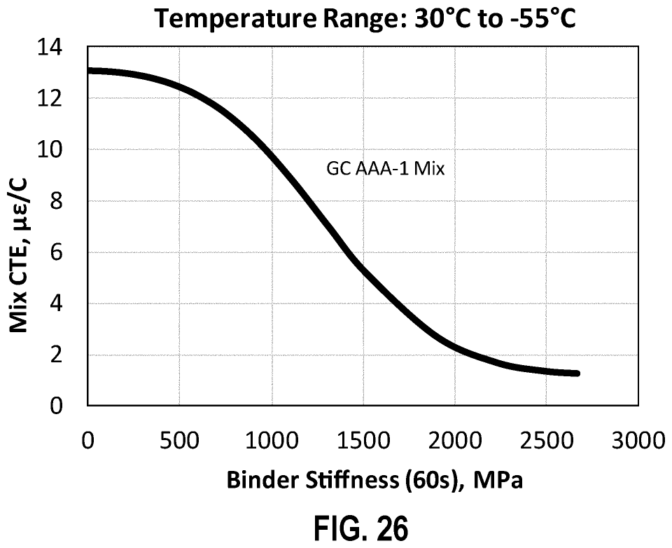

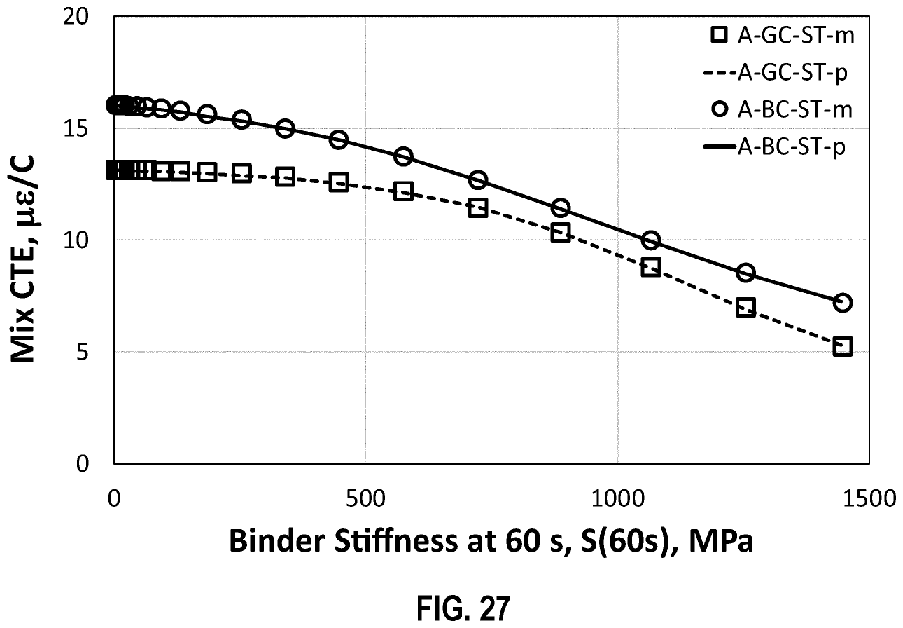

Further measured and plotted data is shown in FIGS. 25-27. FIG. 25 is a graph plotting mixture CTE against temperature for the two mixtures including AAA-1 binder, and including unaged binder master curve from −15° C. and −21° C. BBR (for AAA-1 binder). FIG. 26 is then a plot of binder stiffness versus mixture CTE (in a temperature range of 30° C. to −55° C., for the GC AAA-1 mixture). CTE of the mixture and stiffness appear to have a causal relationship. For all 8 mixtures, T_{g} occurs near binder S(60 s)=1,000 MPa. And FIG. 27 shows mixture CTE as a sigmoid function of stiffness at 60 s.

Following these results, the possible roles of mixture components during cooling were considered. These include that aggregates contract and possibly re-orient slightly; the binder may flow (relax) and fracture; the bulk binder contracts; the interfacial binder resists thermal deformation and can be a source of transition behavior (above Tg); and air provides room for the binder to flow in and out. Following this, the present inventor considered various mixture CTE predictions.

The first of these was a mixture CTE prediction by α_{agg}, α_{b}(T) and S(60 s). The equation used was:

$$CTE = a 1 \cdot {α}_{agg} \cdot {V}_{a g g} + b 1 \cdot {α}_{b} \cdot {V}_{b} + \{ a 2 \cdot {α}_{a g g} \cdot {V}_{agg} + b 2 \cdot {α}_{b} \cdot {V}_{b} \} / [ 1 + exp \{ \frac{S ( 60 s ) - c}{d} \} ]$$

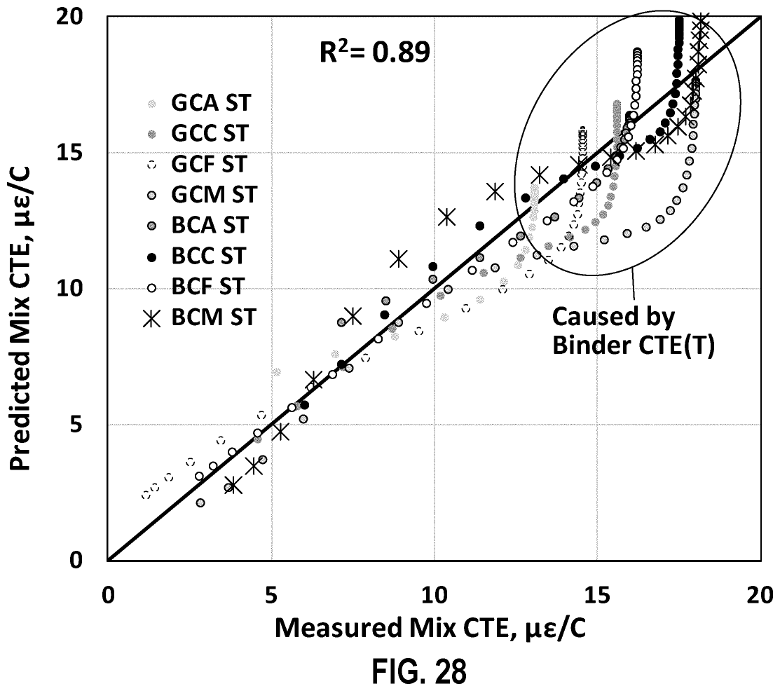

Numerical values were as follows: a1=0.166; a2=0.847; b1=0.06; and b2=0.469. Then, for AAA-1, c=1559 and d=961; for AAC-1, c=1698 and d=369; for AAF-1, c=1180 and d=482; and for AAM-1, c=1208 and d=223. Results of the predicted mixture CTE (calculated in this manner) versus the measured mixture CTE (for each of the eight mixtures) are shown in the graph of FIG. 28.

The second manner of prediction was a mixture CTE prediction by α_{agg}, α₁ and S(60 s). The equation used was:

$$CTE = 0.64 \cdot \{ {α}_{a g g} \cdot {V}_{a g g} + {α}_{l} \cdot {V}_{b} \} / [ 1 + exp \{ \frac{S ( 60 s ) - 1069}{602} \} ]$$

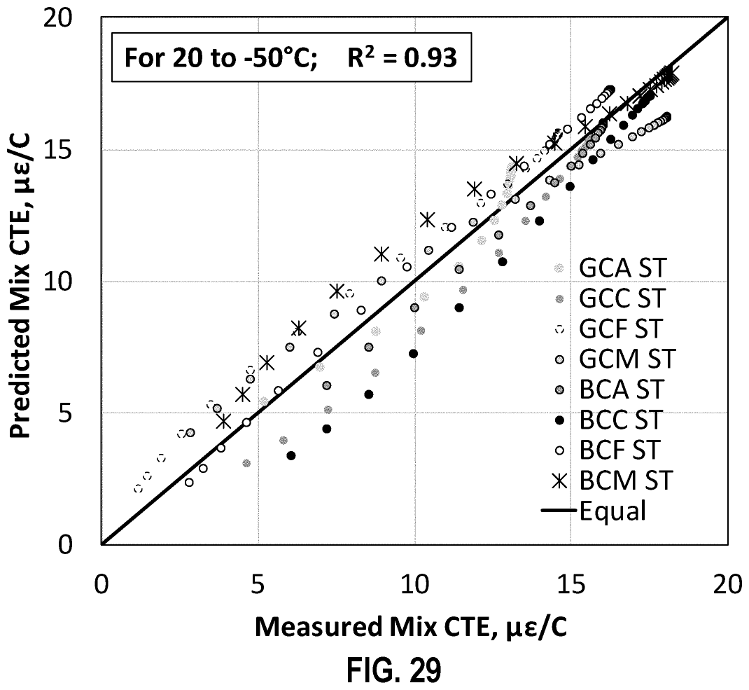

Using unaged binder S(60 s) and constant binder αₗ, the maximum mixture CTE is 0.64(α_{agg}V_{agg}+α₁V_{binder}). And the minimum mixture CTE is 0.025. Results for each of the eight mixtures are shown in FIG. 29.

The third manner of prediction was a second mixture CTE prediction by α_{agg}, a₁ and S(60 s). The equation used was:

$$CTE = 0. 662 \cdot \{ {α}_{a g g} \cdot {V}_{a g g} + {α}_{l} \cdot {V}_{b} \} / [ 1 + exp \{ \frac{S ( 60 s ) - c}{d} \} ]$$

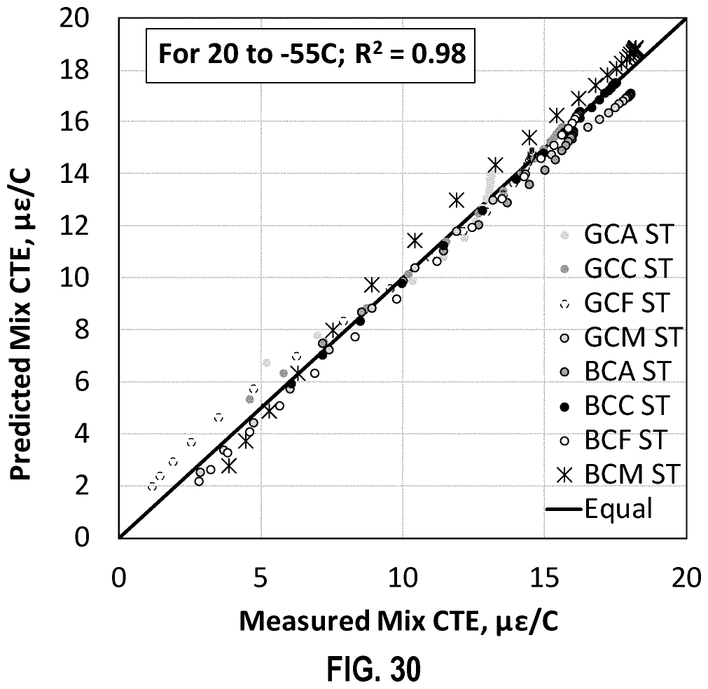

Numerical values for the mixtures were: For AAA-1, c=1150 and d=798; for AAC-1, c=1326 and d=766; for AAF-1, c=861 and d=660; and for AAM-1, c=908 and d=476. Results for each of the eight mixtures are shown in FIG. 30.

The present inventor then developed a model prediction for the effects that binder content might have. Equation is as follows:

$$CTE = 0.6 \; 62 \cdot \{ {α}_{a g g} \cdot {V}_{a g g} + {α}_{l} \cdot {V}_{b} \} / [ 1 + exp \{ \frac{S ( 60 s ) - 861}{660} \} ]$$

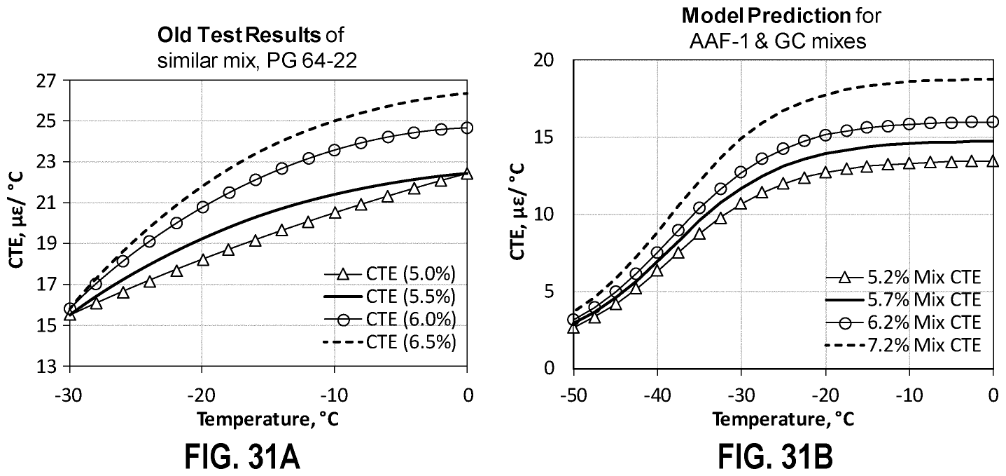

In that regard, FIG. 31A shows test results of CTE against temperature for PG 64-22 binder, and FIG. 31 B shows the model prediction for AAF-1 and GC mixtures. The magnitude of CTE changes due to binder content are similar. The current model using binder S(60 s) cannot estimate the possible effect of the binder content on CTE transition (T_{g} and R).

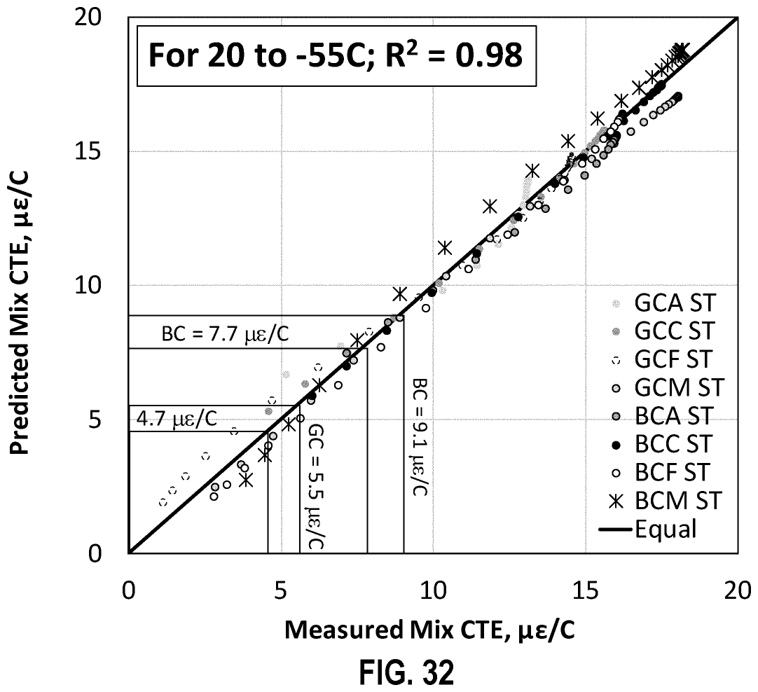

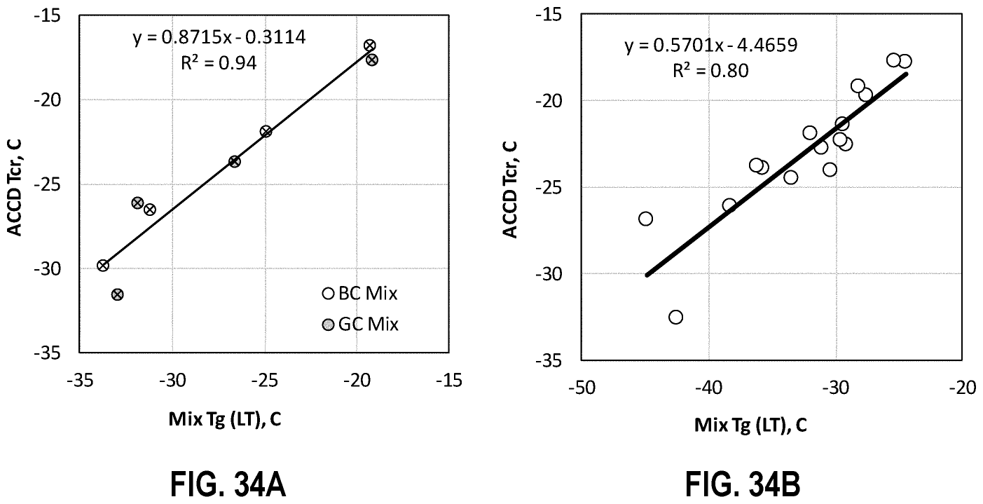

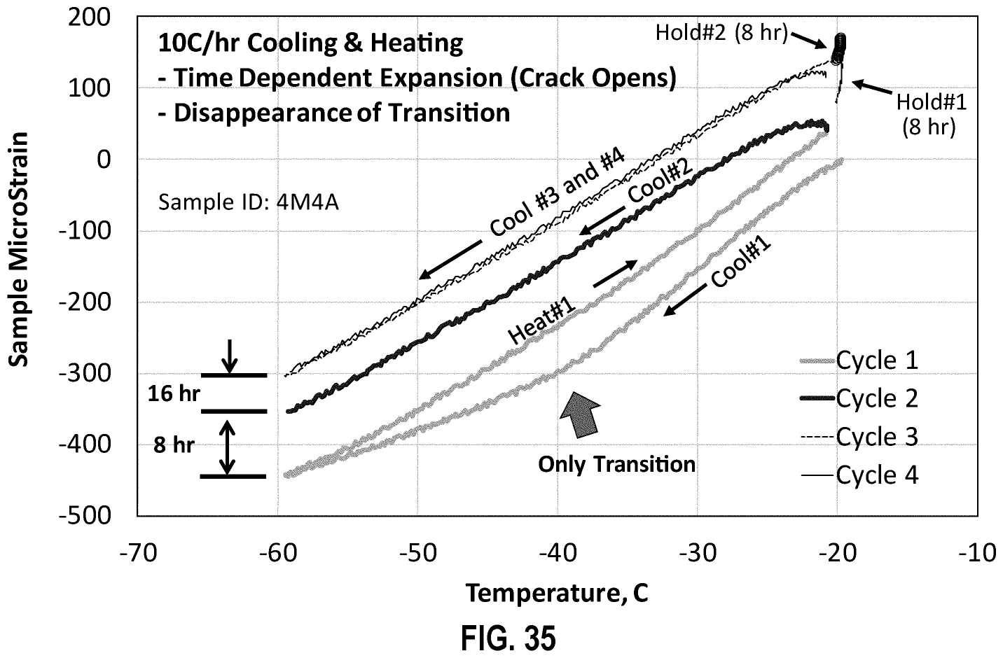

All predicted mixture CTEs plotted against measured mixture CTEs are shown in the graph of FIG. 32. And it was determined that mixture CTE can be smaller than aggregate CTE if there is local failure (see FIG. 33, which shows internal damage—internal crack of binder—that can occur during cooling, with the cracking causing volume expansion; this has a near zero CTE). Thus, the present inventor concluded that mixture transition temperature (Tₜᵣ) must correlate with cracking temperature (T_{cr}). FIGS. 34A and 34B confirm that this is the case. The graphs of these figures plot cracking temperature (as measured with ACCD) against transition temperature. Additional evidence for this conclusion is shown in FIG. 35, which shows thermal strain versus temperature plot for asphalt mixture sample cooled to −60° C. and warmed to −20° C. repeatedly for four cycles. As can be seen, on the first cooling cycle, the transition was observed near −38° C. On subsequent temperature cycles either cooling or heating cycles, no transition was observed. For the test shown in FIG. 35, sample was warmed up only to −20° C. where the temperature is too low for healing to take place. After the first cooling and transition (cracking), the thermal contraction of sample recovered and the sample strain slowly increased over about 24 hour period.

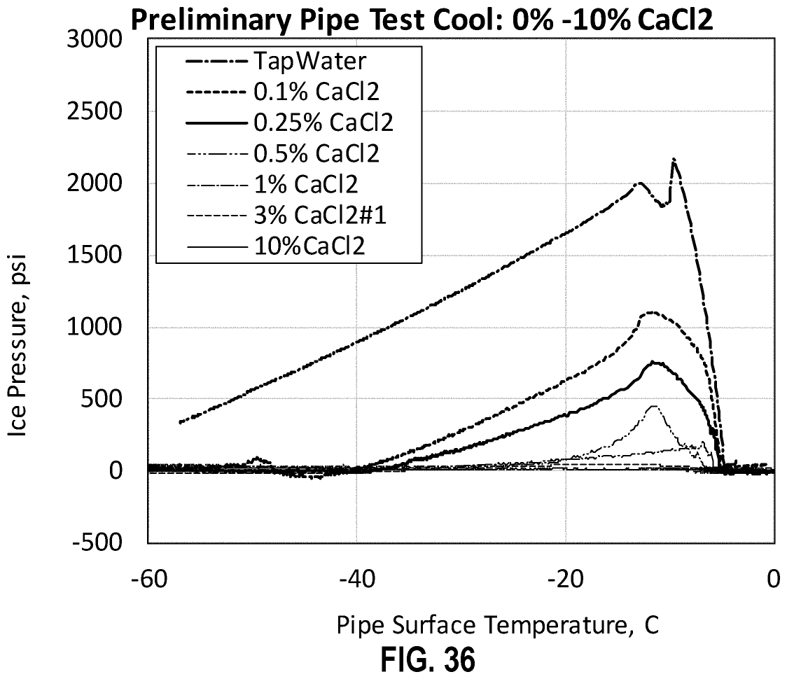

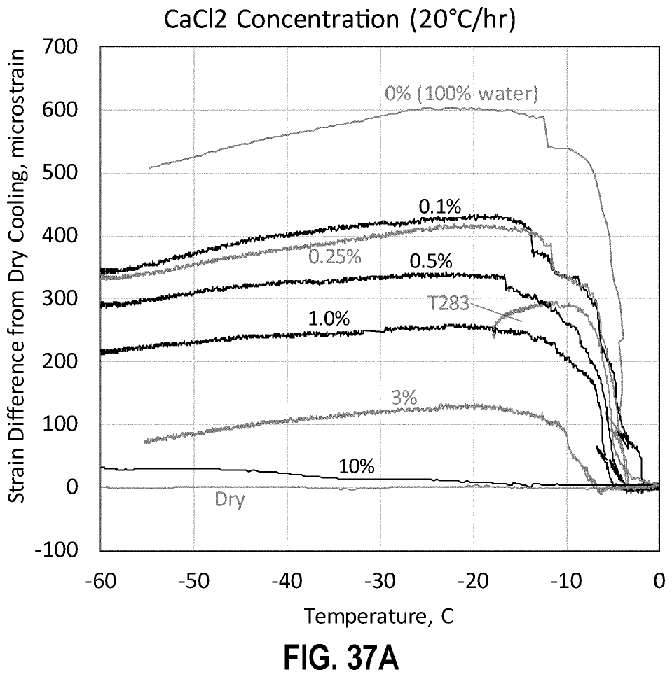

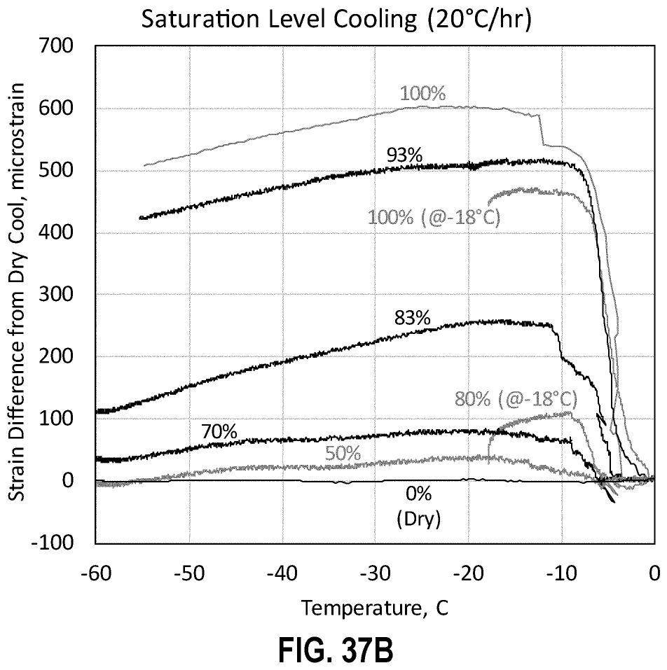

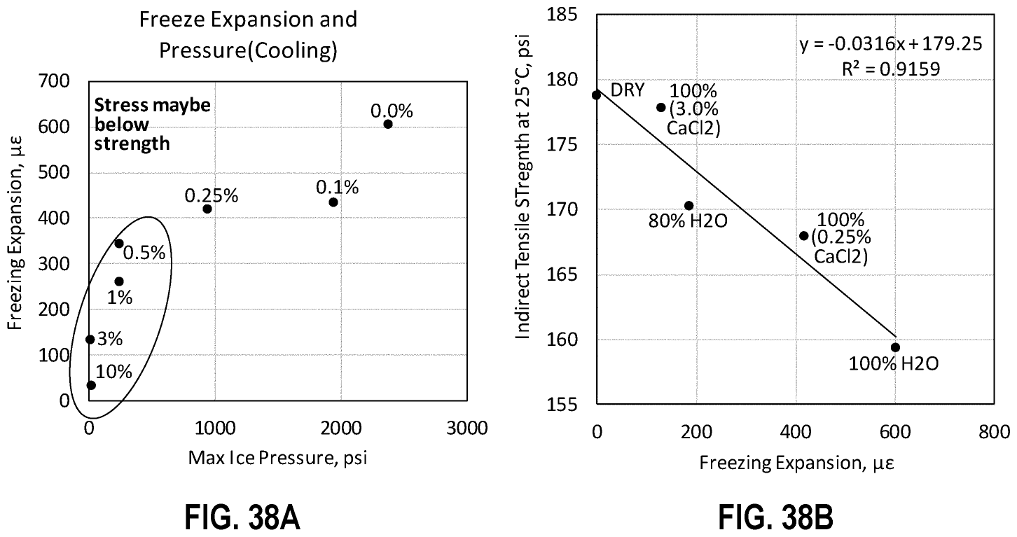

The present inventor then investigated the effect of moisture freezing and thawing (saturation and CaCl₂) on asphalt. Samples were loaded in the CTE device, and an open ended pipe for ice pressure measurement was included. An open tube test was performed for the effects of salt concentration. Results are show in the graph of FIG. 36. CaCl₂ lowers ice growth pressure due to the presence of brine pockets. [Concentration >3% CaCl₂; Ice pressure <HMA ITS or no damage]. FIGS. 37A and 37B are graphs showing the effect of salt (37A) and saturation (37B) on HMA damage (with freeze expansion measured by the CTE device). FIG. 38A is a graph showing freeze expansion versus ice pressure, and FIG. 38B shows indirect tensile strength against freezing expansion after one freezing cycle.

Conclusions

Based on the above experiments, the present inventor determined the following: asphalt mixture CTE is significantly affected by binder grade, RAP/RAS, aggregate type and size, binder content, and aging. Thus, asphalt mixture CTE is important and the theoretical calculations indicated 7° C. or more change (7° C.˜10° C. change) in cracking temperature due to CTE alone.

Based on the one dense gradation and eight mixture test results described above, a new CTE model is now proposed by the present inventor:

$$CTE = 0.64 \cdot \frac{\{ {α}_{a g g} \cdot {V}_{a g g} + {α}_{b} \cdot {V}_{binder} \}}{[ 1 + e x p \{ \frac{S ( 60 s ) - 1069}{602} \} ]}$$

Further, based on the one dense gradation and eight mixture test results described above: (1) under cooling, some of volume changes of aggregate and asphalt binder are not reflected in mixture volume change (above Tₜᵣ, the unaccounted volume is 40-50% and, below Tₜᵣ, significantly more); (2) it appears that binder stiffness is related to thermo-volumetric transition of asphalt mixture above Tₜᵣ; (3) it appears that internal cracking is related to thermo-volumetric transition of asphalt mixture below Tₜᵣ; and (4) mixture Tₜᵣ are highly correlated to the single event low temperature cracking.

While the various aspects of the present invention have been disclosed by reference to the details of various embodiments of the invention, it is to be understood that the disclosure is intended as an illustrative rather than in a limiting sense, as it is contemplated that modifications will readily occur to those skilled in the art, within the spirit of the invention and the scope of the appended claims.

What is claimed is:

1. A method of predicting or determining temperature for thermal cracking of a composite material, comprising:
reducing the temperature of a composite material along a range of temperatures from a first temperature to a second temperature;
measuring dimensional changes in said composite material at a plurality of temperature points along said range of temperatures to generate a curve related to values for the coefficient of thermal expansion for said composite material; and
determine the transition temperature for said composite material, said transition temperature being at the intersection of two asymptotes of said curve;
wherein said transition temperature correlates to the thermal cracking temperature of said composite material.

2. The method of claim 1, wherein the composite material is an asphalt mixture.

3. The method of claim 1, wherein the first temperature is about 20° C. and the second temperature is about −60° C.

4. The method of claim 3, wherein reducing the temperature of said composite material is performed at a rate chosen from 10° C. per hour and 20° C. per hour.

5. The method of claim 1, wherein measuring dimensional changes in said composite material further includes obtaining a first measurement of dimensional change and a second measurement of dimensional change.

6. The method of claim 5, wherein said first measurement of dimensional change and said second measurement of dimensional change are taken in a manner substantially perpendicular to one another.

7. The method of claim 6, wherein said first measurement and said second measurement are taken by a first linear variable differential transducer and a second linear variable differential transducer.

8. The method of claim 1, further comprising obtaining a plurality of temperature measurements of said sample.

9. The method of claim 8, wherein said plurality of temperature measurements of said sample includes a first measurement taken at an exterior surface of said sample, and a second measurement taken at the interior of said sample.

10. The method of claim 9, wherein said plurality of temperature measurements further comprises a third measurement taken of an interior space of a chamber, said composite material being positioned in said interior space.

11. The method of claim 10, wherein said positioning of said composite material within said interior space occurs within a frame disposed within said interior space.

12. The method of claim 11, wherein said plurality of temperature measurements further comprises a fourth measurement taken of said frame.

13. The method of claim 8, wherein said plurality of temperature measurements are each taken at sixty second intervals.
